SEQUENCE LISTING (28 sequences)

SEQ ID NO: 1 - DNA, 681 bp, Homo sapiens
ccaaaggctcagagaagacatgctctgaacatactcaaagtaactgacactggaaaaggtaacagaggtgtgaaaatcttaccatagtagatgtgtgagtgggctgggggcaggtgaattccagagaaatgtggcactaaatgtcatgaagctgactctgtattataccagagtggcctccagattgctttcacaactgggtagttcatcagctaatgtgattctccaaactttaaatgattagaaaactggggaaaaatgtagaaaaccagaggaaaacactccttctaatccaaatataaattcttcacttcttttcaatgttcttccaggagaaggattcgttgggctctttgccttctgcttttatttctgtgcaagggtttatgcaagaggtacttgagaatgctgtactgtagagctttgtttctcatgggaacacacggggaagtggaaaaccctccaaattgtttggtgagaaaacataacatttatccctttctttggtggggtgagtcaagtggtagggactagaattcaggtcctcaatgggcatataaatatgtgtgtgcaaaagcagccatcacactttgtatggcaagtggaaccactggcttggtggattttgctagatttttctgatttttaaactcctgaaaaatatcccagataactgtcatg

SEQ ID NO: 2 - DNA, 366 bp, Homo sapiens
cccagataactgtcatgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtgacatcaagataaagagcggaggtggatggggatggaagatgatgctcctatcctccctgcctgaaacctgt

SEQ ID NO: 3 - DNA, 523 bp, Mus musculus
aggtgacagcgagcagaactattgacaccgtgattttgttgggctttctgactgcattccaaagtcccggaaaacatcacaggcaccagctatgaagctggtatctatctttctgctggtgaccattggtatttgtggttattctgccactgcccttctcatcaaccgtctccctgttgttgacaaattacctgtacctttggacgacattattccctcatttgatcccttgaagatgcttctgaaaaccctgggcatttctgtagaacatctggtgacaggactgaagaagtgtgtggacgagctgggaccagaggcttccgaggccgtgaagaagcttctggaggctctttcacacctggtataaaatcttcataaagagatttaaggaaggagtatgaaaagaaggatttgctcactctggctggctggatctctcattctatcatttgtaaactgaatgtcccagagtttaaggagtctagaaaagtatgaataaagcaatgaaaagaaaaaaaaaaaa

SEQ ID NO: 4 - DNA, 20 bp, Artificial Sequence
  Oligonucleotide primer
gtagaacatctggtgacagg

SEQ ID NO: 5 - DNA, 20 bp, Artificial Sequence
  Oligonucleotide primer
cagccagagtgagcaaatcc

SEQ ID NO: 6 - DNA, 20 bp, Artificial Sequence
  Oligonucleotide primer
tccctgggagaagcctttgc

SEQ ID NO: 7 - DNA, 20 bp, Artificial Sequence
  Oligonucleotide primer
ggagtccctgggatatgcac

SEQ ID NO: 8 - DNA, 20 bp, Artificial Sequence
  Oligonucleotide primer
gactgcattccaaagtcccg

SEQ ID NO: 9 - DNA, 20 bp, Artificial Sequence
  Oligonucleotide primer
ctacagacaccaaagcctcc

SEQ ID NO: 10 - DNA, 20 bp, Artificial Sequence
  Oligonucleotide primer
aaggaggggttcgaggagac

SEQ ID NO: 11 - DNA, 22 bp, Artificial Sequence
  Oligonucleotide primer
gtaatacgactcactatagggc

SEQ ID NO: 12 - DNA, 20 bp, Artificial Sequence
  Oligonucleotide primer
tgcctgtgatgttttccggg

SEQ ID NO: 13 - DNA, 38 bp, Artificial Sequence
  Oligonucleotide primer
ggtgccagaacatttctctacgggagactacttctgtg

SEQ ID NO: 14 - DNA, 38 bp, Artificial Sequence
  Oligonucleotide primer
cacagaagtagtctcccgtagagaaatgttctggcacc

SEQ ID NO: 15 - DNA, 42 bp, Artificial Sequence
  Oligonucleotide primer
gtggaaaacccttcctaatgtttagttaggaagattgccctg

SEQ ID NO: 16 - DNA, 42 bp, Artificial Sequence
  Oligonucleotide primer
cagggcaatcttcctaactaaacattaggaagggttttccac

SEQ ID NO: 17 - DNA, 29 bp, Artificial Sequence
  Oligonucleotide primer
aaaggatcctataggaaagcattcctctc

SEQ ID NO: 18 - DNA, 29 bp, Artificial Sequence
  Oligonucleotide primer
aaactcgagtgatggctgcttttcctcag

SEQ ID NO: 19 - DNA, 350 bp, Mus musculus
tataggaaagcattcctctcaaacccaacagaaagccttcacatctgctttagtgttcttccagggggaaatcctttgccgtctgtcatcatttctatgctagggcttgggaaagaactacttgagactacttctgtgaacagctttgtttctcacgagaatgtgatgaaaagtggaaaacccttcagaatgtttagttaggaagattgccctgcatgctcttctttgctggggtaagtcaacaggcagtggttgaaattcaggtcttcagtgcatatattagtacctctgaggaaaagcagccatcaggtgacagcgagcagaactattgacaccgtgattttgttggg

SEQ ID NO: 20 - protein, 91 aa, Mus musculus
MKLVSIFLLVTIGICGYSATALLINRLPVVDKLPVPLDDIIPSFDPLKMLLKTLGISVEHLVTGLKKCVDELGPEASEAVKKLLEALSHLV

SEQ ID NO: 21 - protein, 104 aa, Mus musculus
MKLTTTFLVLCVALLSDSGVAFFMDSLAKPAVEPVAALAPAAEAVAGAVPSLPLSHLAILRFILASMGIPLDPLIEGSRKCVTELGPEAVGAVKSLLGVLTMFG

SEQ ID NO: 22 - protein, 93 aa, Homo sapiens
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 23 - protein, 104 aa, Homo sapiens
MKLAALLGLCVALSCSSAAAFLVGSAKPVAQPVAALESAAEAGAGTLANPLGTLNPLKLLLSSLGIPVNHLIEGSQKCVAELGPQAVGAVKALKALLGALTVFG

SEQ ID NO: 24 - DNA, 25 bp, Artificial Sequence
  Oligonucleotide primer
cctccagattgctttcacaactggg

SEQ ID NO: 25 - DNA, 25 bp, Artificial Sequence
  Oligonucleotide primer
caaagtgtgatggctgcttttgcac

SEQ ID NO: 26 - protein, 96 aa, Mus musculus
MKIAITITVVMLSICCSSASSDICPGFLQVLEALLMESESGYVASLKPFNPGSDLQNAGTQLKRLVDTLPQETRINIMKLTEKILTSPLCKQDLRF

SEQ ID NO: 27 - protein, 93 aa, Homo sapiens
MKLLMVLMLAALSQHCYAGSGCPLLENVISKTINPQVSKTEYKELLQEFIDDNATTNAIDELKECFLNQTDETLSNVEVFMQLIYDSSLCDLF

SEQ ID NO: 28 - protein, 95 aa, Rattus norvegicus
MKLVFLFLLVTIPICCYASGSGCSILDEVIRGTINSTVTLHDYMKLVKPYVQDHFTEKAVKQFKQCFLDQTDKTLENVGVMMEAIFNSESCQQPS

(19) United States
(12) Patent Application Publication
(10) Pub. No.: US 2004/0234982 A1
(43) Pub. Date: Nov. 25, 2004

(54) Human ugrp (uteroglobin-related protein) 1 promoter and its use

(76) Inventors: Shioko Kimura, Bethesda, MD (US); Tomoaki Niimi, Bethesda, MD (US)

Correspondence Address: KLARQUIST SPARKMAN, LLP, 121 SW SALMON STREET, SUITE 1600, PORTLAND, OR, 97204, US

(21) Appl. No.: 10/480,693

(22) PCT Filed: Jun. 18, 2002
(86) PCT No.: PCT/US02/19456

Related U.S. Application Data
(60) Provisional application No. 60/299,828, filed on Jun. 20, 2001.

(51) Int. Cl. C07K 14/705; C12Q 1/68; C07H 21/04

(52) U.S. Cl. 435/6; 435/69.1; 435/325; 530/350; 536/23.5; 435/320.1

(57) ABSTRACT

The nucleic acid sequence of mammalian Uteroglobin Related Protein (e.g., UGRP1 genes are disclosed. Specifically the mouse and the human UGRP1 promoters are disclosed herein. Vectors are disclosed that include these promoters, and these promoters operably linked to a heterologous nucleic acid sequence. Host cells are disclosed that are transformed with theses UGRP1 promoter sequences. A method is disclosed for determining the diagnosis or prognosis of a respiratory disorder in a subject, utilizing the UGRP1 promoter sequence. In one embodiment, the disorder is asthma. In another embodiment, the subject is a human, and the presence of a polymorphism in the UGRP1 promoter sequence is used to diagnose, or determine the prognosis of a respiratory disorder. One specific, non-limiting example of a polymorphism disclosed herein is a polymorphism at position −112 of the UGRP1 promoter.

### FIELD

The present invention is generally related to prediction and diagnosis of disease states, for example prediction of a predisposition of a subject to a respiratory disorder, such as asthma.

### BACKGROUND

Asthma (sometimes referred to as reactive airway disease) is a condition of the respiratory tract characterized by widespread, reversible narrowing of the airways (bronchoconstriction) and increased sensitivity (hyperresponsiveness) of the airways to a variety of stimuli. The familiar symptomology of asthma (e.g., coughing, wheezing, chest tightness, dyspnea) is caused by airway smooth muscle contraction, increased bronchial mucus secretion, and inflammation. Though seldom fatal, asthma has been estimated to affect 10-20% of school-aged children around the world, and hospital admissions for asthma in children have increased dramatically in recent years, one survey for the United States indicating that hospital admissions for children under 15 with asthma increased by at least 145% between 1970 and 1984 (See, Sears, in Asthma as an Inflammatory Disease, O'Byrne, (ed.), Marcel Dekker, Inc.; New York, 1990, pp. 15-48). Overall, it is estimated that 10 million Americans (4% of the population) have asthma, and some $4 billion is spent in treatment per year (Altman, New York Times, The Doctor's World, Mar. 26, 1991).

The inflammatory response in asthma is typical for tissues covered by a mucosa and is characterized by vasodilation, plasma exudation, recruitment of inflammatory cells such as neutrophils, monocytes, macrophages, lymphocytes and eosinophils to the sites of inflammation, and release of inflammatory mediators by resident tissue cells (e.g., mast cells) or by migrating inflammatory cells. In allergen-induced asthma, sufferers often exhibit a dual response to exposure to an allergen—an “early phase” response beginning immediately after exposure and lasting until 1-2 hours after exposure, followed by a “late phase” response beginning about 3 hours after exposure and lasting sometimes until 8-10 hours or longer after exposure late phase response in allergen-induced asthma and persistent hyperresponsiveness have been associated with the recruitment of leukocytes, and particularly eosinophils, to inflamed lung tissue.

The causes of asthma are not completely understood, however the study of agents that trigger acute asthmatic episodes supports the theory that asthma is an immunological reaction by a subject in response to specific allergens of the subject's environment. These “triggers” exacerbate asthma by causing transient enhancement of airway hyperresponsiveness. Triggers that have been found to induce airway hyperresponsiveness include inhaled allergens, inhaled low molecular weight agents to which the subject has become sensitized (e.g., by occupational exposure), viral or mycoplasma respiratory infections, and oxidizing gases such as ozone and nitrogen dioxide. These “inducing” triggers can be distinguished from “inciting” triggers of bronchospastic episodes which include exercise, cold air, emotional stress, pharmacological triggers, and inhaled irritants. The common feature of inducing triggers is that they are associated with airway inflammation; inciting triggers produce smooth muscle contractions (bronchospasms) which depend on the underlying degree of hyperresponsiveness, rather than increasing airways responsiveness themselves (see, Cockcroft, in Asthma as an Inflammatory Disease, O'Byrne (ed.), Marcel Dekker, Inc.; New York, 1990, pp. 103-125).

Asthma is strongly familial, and is believed to be a result of an interaction between genetic and environmental factors. The discovery of genetic factors which predispose to asthma allows better classification of disease subtypes with distinct clinical courses and responses to therapy (Moffatt and Cookson, Int. Arch. Allergy Immunol. 116:247-252, 1998). In addition, asthma and related allergic diseases may become preventable once the recognition of children at risk is possible. Eventually, the identification of the genes involved in asthma may lead to new pharmacological treatments.

Genes are known to predispose to asthma because the sequences contain polymorphisms that alter gene function. Several candidate genes have already been identified that are linked to susceptibility to asthma. For example, there is evidence for one polymorphism associated with asthma in the 5q cytokine cluster (Postma et al., New Engl. J Med. 333:894, 1995), and one polymorphism associated with asthma on chromosome 12q (Bames et al. Genomics 37:41-50, 1996). In addition, several other genetic loci have been described that are associated with asthma or atopy (Moffat and Cookson, Int. Arch. Allergy Immunol. 116:247-252, 1998). However, a need remains to find more genetic tools useful in the diagnosis of asthma and that, may also be used to determine the clinical course of therapy.

### SUMMARY

The nucleic acid sequence of mammalian Uteroglobin Related Protein (e.g., UGRP1) genes are disclosed. Specifically the mouse and the human UGRP1 promoters are disclosed herein. Vectors are disclosed that include these promoters, and these promoters operably linked to a heterologous nucleic acid sequence. Host cells are disclosed that are transformed with these UGRP1 promoter sequences.

A method is disclosed for determining the diagnosis or prognosis of a respiratory disorder in a subject, utilizing the UGRP1 promoter sequence. In one embodiment, the disorder is asthma. In another embodiment, the subject is a human, and the presence of a polymorphism in the UGRP1 promoter sequence is used to diagnose, or determine a propensity toward developing, or the prognosis of, a respiratory disorder. One specific, non-limiting example of a polymorphism disclosed herein is a polymorphism at position—112 of the UGRP1 promoter.

### BRIEF DESCRIPTION OF THE DRAWINGS

FIG. 1A is a schematic representation of the mouse UGRP1 gene structure, showing the organization of exons and introns, and three types of transcripts. Solid boxes represent exons. The translation initiation and termination codons including one within the intron 2 are indicated. Sequences in intron 2 that are retained in type B and C transcripts are shown by a thick line. A thin jagged line shows sequences that are spliced out in mature mRNAs. The size of each transcript is given on the right. Arrows indicate the positions of primers used for reverse transcriptase polymerase chain reaction (RT-PCR) analysis (P1-P5). FIG. 1B is a schematic diagram of an alignment of UGRP1 type A amino acid sequence (SEQ ID NO:20). UGRP1 is aligned with mouse uteroglobin/CCSP (mUG/CCSP) (SEQ ID NO:26) (Margraf et al., Am J Respir Cell Mol Biol 9:231-38, 1993), human mammaglobin A (hMAM-A) (SEQ ID NO:27) (Watson et al., Cancer Res 56:860-65, 1996), and rat prostatein C3 (rPSC3) (SEQ ID NO:28) (Parker et al., J Biol Chem 258:12-15, 1983). Identical residues are shown in shaded box. The asterisks indicate the conserved cysteine and lysine residues present in uteroglobin/CCSP gene family (Mukherjee et al., Cell Mol Life Sci 55:771-87, 1999). The antiflammin region in the uteroglobin/CCSP (Mukherjee en al., Cell Mol Life Sci 55:771-87, 1999) and the predicted UGRP1 signal sequence are shown by a bracket and a line above the alignment, respectively. FIG. 1C is the UGRP1 type A amino acid sequence (SEQ ID NO:20) aligned with the sequence of mouse UGRP2 (SEQ ID NO:21), and human UGRP1 (SEQ ID NO:22) and 2 (SEQ ID NO:23). The identical and conserved residues are shown in black and shaded box, respectively.

FIG. 2 is the sequence of the mouse UGRP1 gene promoter (SEQ ID NO:19). Arrowheads indicate the position of −242, −190, −147, −67 and −18 deletion constructs used in transfection analyses. The minimal T/EBP/NKX2.1 binding consensus sequences (CTNNAG) (Bohinski et al., Mol Cell Biol 14:5671-681, 1994) are shown in bold. The TATA sequence is boxed. Bent arrow with +1 indicates the major transcription start site. The protected regions I through IV are underlined.

FIGS. 3A and B are schematic diagrams of transfection analyses of mouse UGRP1 gene. FIG. 3A is a schematic illustration of mutant constructs. An asterisk indicates a base change. FIG. 3B is a schematic diagram of deletion analyses of the mouse UGRP1 gene promoter. The relative luciferase activity of NCI-H441 cells transiently transfected with the indicated deletion or mutant constructs is shown based on the activity obtained with the basic vector as 1 in the presence of co-expressed pCMV4-T/EBP/NKX2.1 (black bars) or pCMV4 (white bars). Data are the mean value of at least three experiments (duplicate samples)±S.D. FIG. 3C is a deletion analysis of the mouse UGRP1 gene promoter in HeLa cells. The relative luciferase activity was expressed as described in B.

FIG. 4 is a diagram showing the location of the oligonucleotide sequences used as probes in electrophoretic mobility shift analysis; probe I: −200 to −173 bp in mouse UGRP1 gene promoter (SEQ ID NO:19), probe II: −136 to −113 bp in mouse UGRP1 gene promoter (SEQ ID NO:19), probe I mut: T/EBP/NKX2.1 binding site mutated in probe I, probe II mut: T/EBP/NKX2.1 binding site mutated in probe II. Oligo C is an oligonucleotide taken from the rat thyroglobulin promoter, which had been identified as T/EBP/NKX2.1 binding site (Civitareale et al., EMBO J 8:2537-542, 1989). Putative T/EBP/NKX2.1 consensus and the mutated sequences are shown in bold and underlined.

FIG. 5 shows the human UGRP1 gene promoter (SEQ ID NO:1) and gene structure. FIG. 5A is the sequence of the human UGRP1 gene promoter (SEQ ID NO:1). FIG. 5B is the structure of the gene and the sequences at the exon-intron boundaries. Sequences are shown in upper (exon) and lower (intron) case letters. Splicing donor and acceptor consensus sequences are shown in bold.

FIG. 6 shows UGRP1 expression. Human multiple tissue expression array was hybridized with ³²P-labeled human UGRP1 cDNA probe. RNA sources are as follows: A1=whole brain; A2=amygdala; A3=caudate nucleus; A4=cerebellum; A5=cerebral cortex; A6=frontal lobe; A7=hippocampus; A8=medulla oblongata; B1=occipital lobe; B2=putamen; B3=substantia nigra; B4=temporal lobe; B5=thalamus; B6=subthalamic nucleus; B7=spinal cord; C1=heart; C2=aorta; C3=skeletal muscle; C4=colon; C5=bladder; C6=uterus; C7=prostate; C8=stomach; D1=testis; D2=ovary; D3=pancreas; D4=pituitary gland; D5=adrenal gland; D6=thyroid gland; D7=salivary gland; D8=mammary gland; E1=kidney; E2=liver; E3=small intestine; E4=spleen; E5=thymus; E6=peripheral leukocyte; E7=lymph node; E8=bone marrow; F1=appendix; F2=lung; F3=trachea; F4=placenta; G1=fetal brain; G2=fetal heart; G3=fetal kidney; G4=fetal liver; G5=fetal spleen; G6=fetal thymus; and G7=fetal lung.

FIG. 7 is a series of schematic diagrams showing the human UGRP1 gene promoter analysis and −112G/A polymorphism. FIG. 7A is the sequence of the promoter region (−209 to +96 bp of SEQ ID NO:1) used for transfection analysis. Numbers indicate nucleotide positions relative to the major transcription start site, marked by a bent arrow (+1). The nucleotide −112 is a polymorphic site; polymorphic G/A nuclcotide is shown in bold. TATA box is boxed and ATG initiation codon is shown in bold. FIG. 7B is the DNA sequence of human UGRP1 gene from −209 to +85 bp that was inserted into pGL3-Basic luciferase vector. Polymorphic Nucleotide at −112 bp is indicated. FIG. 7C is a graph of the results from reporter gene assays of human UGRP1 gene promoter constructs. Relative luciferase activities of constructs harboring the human UGRP1 gene sequence from −209 to +85 bp, with either G (−112G) or A (−112A) at −112 bp, were compared in transient transfection studies using NCI-H441 cells. Luciferase activities are shown based on the activity obtained with pGL3-Basic vector (GL3) as 1. The constructs were tested in duplicate in four independent experiments. Values represent the mean±standard deviations.

FIG. 8 is an electrophoretic mobility shift analysis of the −112G/A polymorphic site. FIG. 8A is the oligonucleotide sequences containing G (−112G) or A (−112A) at −112 bp, used as probe or competitor in the electrophoretic mobility shift analysis. The consensus sequence for C/EBP obtained by Transcription Factor Search is underlined. FIG. 8B is a graph of the results obtained in the competition analysis. In these studies, specific DNA-protein complex formed between a nuclear protein in NCI-H441 cells and ³²P-labled −112G fragment was subjected to competition analysis in the presence of increasing concentrations (0.25- to 9-fold) of unlabeled −112G or −112A oligonucleotide as competitor. Band intensity was quantitated using Phospholmager and ImageQuant programs (Molecular Dynamics, Inc., Sunnyvale, Calif.). Note that the affinity of specific DNA-protein complex formation is about 2-fold higher with the −112G oligonucleotide than with the −112A oligonucleotide.

### DETAILED DESCRIPTION OF SPECIFIC EMBODIMENTS

### Explanation of Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanation of terms is provided:

Abnormal: Deviation from normal characteristics. Normal characteristics can be found in a control, a standard for a population, etc. For instance, where the abnormal condition is a disease condition, such as a respiratory disorder (e.g. asthma), a few appropriate sources of normal characteristics might include an individual who is not suffering from the disease (e.g., osteoporosis), a population standard of individuals believed not to be suffering from the disease, etc.

Likewise, abnormal may refer to a condition that is associated with a disease. The term “associated with” includes an increased risk of developing the disease as well as the disease itself. For instance, a certain abnormality (such as an abnormality in a UGRP1 promoter sequence) can be described as being associated with a respiratory disorder such as asthma.

An abnormal nucleic acid, such as an abnormal UGRP1 promoter nucleic acid, is one that is different in some manner from a normal (wildtype) nucleic acid. Such abnormality includes but is not necessarily limited to: (1) a mutation in the nucleic acid (such as a point mutation (e.g., a single nucleotide polymorphism) or short deletion or duplication of a few to several nucleotides); (2) a decrease in the amount or copy number of the nucleic acid in a cell or other biological sample (such as a deletion of the nucleic acid, either through selective gene loss or by the loss of a larger section of a chromosome or under expression of the mRNA); and (3) an increase in the amount or copy number of the nucleic acid in a cell or sample (such as a genomic amplification of part or all of the nucleic acid or the overexpression of an mRNA), each compared to a control or standard. It will be understood that these types of abnormalities can co-exist in the same nucleic acid or in the same cell or sample; for instance, a genomic-amplified nucleic acid sequence may also contain one or more point mutations. In addition, it is understood that an abnormality in a nucleic acid may be associated with, and in fact may cause, an abnormality in expression of the corresponding protein.

Abnormal protein expression, such as abnormal UGRP1 expression, refers to expression of a protein that is in some manner different from expression of the protein in a normal (wildtype) situation. This includes but is not necessarily limited to: (1) a mutation in the protein such that one or more of the amino acid residues is different; (2) a short deletion or addition of one or a few amino acid residues to the sequence of the protein; (3) a longer deletion or addition of amino acid residues, such that an entire protein domain or sub-domain is removed or added; (4) expression of an increased amount of the protein, compared to a control or standard amount; (5) expression of an decreased amount of the protein, compared to a control or standard amount; (6) alteration of the subcellular localization or targeting of the protein; (7) alteration of the temporally regulated expression of the protein (such that the protein is expressed when it normally would not be, or alternatively is not expressed when it normally would be); and (8) alteration of the localized (e.g., organ or tissue specific) expression of the protein (such that the protein is not expressed where it would normally be expressed or is expressed where it normally would not be expressed), each compared to a control or standard.

Controls or standards appropriate for comparison to a sample, for the determination of abnormality, include samples believed to be normal as well as laboratory values, even though possibly arbitrarily set, keeping in mind that such values may vary from laboratory to laboratory. Laboratory standards and values may be set based on a known or determined population value and may be supplied in the format of a graph or table that permits easy comparison of measured, experimentally determined values.

Asthma: A clinical syndrome characterized by recurrent episodes of airway obstruction that resolve spontaneously or as a result of treatment. The resolution of the airway obstruction is a feature that distinguishes it from forms of chronic obstructive lung disease. Asthma is also associated with hyperresponsiveness of the airways to a variety of inhaled stimuli; this condition is manifested as an exaggerated bronchoconstrictor response to stimuli that have little or no effect in normal subjects.

Asthma is sometimes referred to as reactive airway disease.

Episodic airway narrowing constitutes an “asthma attack,” and results from obstruction of the airway lumen to airflow. Three distinct pathological processes account for the obstruction: (1) constriction of airway smooth muscle, (2) thickening of airway epithelium, and (3) the presence of liquids within the confines of the airway lumen. It has been hypothesized that constriction of airway smooth muscle is due to the local release of bioactive mediators or neurotransmitters.

During an asthma attack, patients experience shortness of breath accompanied by cough, wheezing, and anxiety. Dyspnea may occur with exercise. In one embodiment, asthma is diagnosed by the presence of at least two symptoms (recurrent cough, wheezing, or dyspnea), and the presence of reversible airflow limitation (15% variability in forced expiratory volume in one second (FEV1), or in peak expiratory flow rate either spontaneously or with an inhaled short-acting beta2-agonist), or increased airway responsiveness to methacholine.

Binding or stable binding (of an oligonucleotide): An oligonucleotide binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional and physical binding assays. Binding may be detected functionally by determining whether binding has an observable effect upon a biosynthetic process such as expression of a gene, DNA replication, transcription, translation and the like.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, one method that is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

cDNA (complementary DNA): A piece of DNA lacking internal, non-coding segments (introns) and transcriptional regulatory sequences cDNA may also contain untranslated regions (UTRs) that are responsible for translational control in the corresponding RNA molecule cDNA is usually synthesized in the laboratory by reverse transcription from messenger RNA extracted from cells.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide remains detectably bound to a target nucleic acid sequence under the required conditions.

Complementarity is the degree to which bases in one nucleic acid strand base pair with the bases in a second nucleic acid strand. Complementarity is conveniently described by percentage, i.e. the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Conservative Variant of a Promoter: A conservative variant of a promoter is a nucleotide sequence that has one or more nucleotide substitutions, so long as the nucleotide sequence still retains the ability to direct transcription of a nucleic acid. In one embodiment, a conservative variant of a promoter that has one or more nucleotide substitutions, wherein the sequence retains the ability to direct transcription at the same level as the sequence without the nucleotide substitutions. One specific, non-limiting example of a conservative variant of a promoter is SEQ ID NO:1 or SEQ ID NO:19, wherein one nucleotide is substituted, and wherein the variant directs transcription of a heterologous nucleic sequence. Another specific, non-limiting example of a conservative variant of a promoter is SEQ ID NO:1 or SEQ ID NO:19, wherein at most five nucleotides are substituted, and wherein the variant directs transcription of a heterologous nucleic sequence. Thus, the conservative variant (such as SEQ ID NO:1 or SEQ ID NO:19) produces transcripts of the heterologous nucleic acid sequences at the same rate or the same absolute level in a cell.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes UGRP1, or a fragment thereof, encompasses both the sense strand and its reverse complement. Thus, for instance, it is appropriate to generate probes or primers from the reverse complement sequence of the disclosed nucleic acid molecules.

Deletion: The removal of a sequence of DNA, the regions on either side of the removed sequence being joined together.

Genomic target sequence: A sequence of nucleotides located in a particular region in the human genome that corresponds to one or more specific genetic abnormalities, such as a nucleotide polymorphism, a deletion, or an amplification. The target can be for instance a coding sequence; it can also be the non-coding strand that corresponds to a coding sequence.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to an URGP1 encoding mRNA, a UGRP1 promoter, or an UGRP1-encoding dsDNA.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of the present invention, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Nucleotide array: Immobilized nucleotide sequences present in a defined pattern on a solid surface. In one embodiment, nucleotide arrays are used to analyze a sample for the presence of gene variations or mutations, or for patterns of gene expression.

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Open reading frame: A series of nucleotide triplets (codons) coding for amino acids without any internal termination codons. These sequences are usually translatable into a peptide.

Ortholog: Two nucleic acid or amino acid sequences are orthologs of each other if they share a common ancestral sequence and diverged when a species carrying that ancestral sequence split into two species. Orthologous sequences are also homologous sequences.

Pharmaceutically acceptable carriers: The pharmaceutically acceptable carriers useful in this invention are conventional. Martin, Remington's Pharmaceutical Sciences, published by Mack Publishing Co., Easton, Pa., 19th Edition, 1995, describes compositions and formulations suitable for pharmaceutical delivery of the nucleotides and proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polymorphism: Variant in a sequence of a gene. Polymorphisms can be those variations (nucleotide sequence differences) that, while having a different nucleotide sequence, produce functionally equivalent gene products, such as those variations generally found between individuals, different ethnic groups, or geographic locations. The term polymorphism also encompasses variations that produce gene products with altered function, i.e., variants in the gene sequence that lead to gene products that are not functionally equivalent. This term also encompasses variations that produce no gene product, an inactive gene product, or increased or decreased gene product. The term polymorphism may be used interchangeably with allele or mutation, unless context clearly dictates otherwise.

Polymorphisms can be referred to, for instance, by the nucleotide position at which the variation exists, by the change in amino acid sequence caused by the nucleotide variation, or by a change in some other characteristic of the nucleic acid molecule that is linked to the variation (e.g., an alteration of a secondary structure such as a stem-loop, or an alteration of the binding affinity of the nucleic acid for associated molecules, such as transcriptional activators, transcriptional repressors, and so forth). By way of example, the polymorphism disclosed herein in the 5′ untranslated region of the UGRP1 gene can be referred to by its location (e.g., −112, based on the numerical position of the variant residue) or by the effect it has on the transcription of UGRP1 (e.g., decrease transcription of UGRP1).

Probes and primers: A probe comprises an isolated nucleic acid attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., in Sambrook et al. (In Molecular Cloning: A Laboratory Manual, CSHL, New York, 1989) and Ausubel et al. (In Current Protocols in Molecular Biology, John Wiley & Sons, New York, 1998).

Primers are short nucleic acid molecules, for instance DNA oligonucleotides 10 nucleotides or more in length, for example that hybridize to contiguous complementary nucleotides or a sequence to be amplified. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other nucleic-acid amplification methods known in the art. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in 5,427,930; and NASBA™ RNA transcription-free amplification, as disclosed in U.S. Pat. No. 6,025,134.

Nucleic acid probes and primers can be readily prepared based on the nucleic acid molecules provided in this invention. It is also appropriate to generate probes and primers based on fragments or portions of these disclosed nucleic acid molecules, for instance regions that encompass the identified polymorphism at position −112 in the UGRP1 promoter sequence.

Methods for preparing and using nucleic acid probes and primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). Amplification primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, for example, a primer comprising 30 consecutive nucleotides of an UGRP1 promoter or flanking region thereof (an “UGRP1 promoter primer” or “UGRP1 promoter probe”) will anneal to a target sequence with a higher specificity than a corresponding primer of only 15 nucleotides. Thus, in order to obtain greater specificity, probes and primers can be selected that comprise at least 20, 25, 30, 35, 40, 45, 50 or more consecutive nucleotides of a UGRP1 promoter nucleotide sequences.

The invention thus includes isolated nucleic acid molecules that comprise specified lengths of the UGRP1 promoter sequence and/or flanking regions. Such molecules may comprise at least 10, 15, 20, 23, 25, 30, 35, 40, 45 or 50 consecutive nucleotides of these sequences or more, and may be obtained from any region of the disclosed sequences. By way of example, the human UGRP1 promoter and gene sequences may be apportioned into about halves or quarters based on sequence length, and the isolated nucleic acid molecules (e.g., oligonucleotides) may be derived from the first or second halves of the molecules, or any of the four quarters. The DNA also could be divided into smaller regions, e.g. about eighths, sixteenths, twentieths, fiftieths and so forth, with similar effect.

In particular embodiments, isolated nucleic acid molecules of the invention comprise or overlap at least one residue position designated as being associated with a polymorphism that is predictive of a respiratory disorder such as asthma. Such polymorphism sites include position −112, such as a G to A transition at position −112.

Promoter: A promoter is an array of nucleic acid control sequences which directs transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Protein: A biological molecule expressed by a gene and comprised of amino acids.

Pulmonary function: The function of the respiratory system, which can be measured through a variety of tests, including, but not limited to measurements of airflow (e.g. spirometry) or arterial blood gases. Measurements of airflow included airflow rate, peak expiratory flow rate (PEFR), forced expiratory volume in the first second (FEV₁), and maximal midexpiratory rate (MMEFR). A decrease in airflow rates throughout the vital capacity is the cardinal pulmonary function abnormality in asthma. Although essential for the diagnosis of asthma, it is not specific, as other obstructive diseases share this feature. The PEFR, FEV₁, and MMEFR are all decreased in asthma. The severity of the attack of asthma can be assessed by objective measurements of airflow.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

Respiratory Disorder: A large variety of abnormalities arising in all the different structures of the body involved with gas exchange. These structures include the lungs, nose, oropharynx, extrapulmonary airways, thoracic cage, and respiratory muscles. Respiratory disorders encompass both acute and chronic diseases. Asthma is one specific, non-limiting example of a respiratory disorder. Other specific non-limiting examples include, but are not limited to, coughs, pneumonia, bronchitis, such as chronic obstructive bronchitis, and emphysema, interstitial lung disease, cystic fibrosis, and lung tumors.

Ribozyme: A ribonucleic acid molecule that has catalytic activity.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a UGRP1 promoter will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443,1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444,1988. In addition, Altschul et al., Nature Genet., 6:119-129, 1994 presents a detailed consideration of sequence alignment methods and homology calculations.

The NCBI Basic Local Alignment Search Tool (BLAST) (Altschul et al., J. Mol. Biol., 215:403-410, 1990.) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website. A description of how to determine sequence identity using this program is available at the NCBI website.

Homologs and variants of a UGRP1 protein are typically characterized by possession of at least 50% sequence identity counted over the full length alignment with the amino acid sequence of a native protein using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 60%, at least 65%, at least 70%, at least 75%, at least 80%, at least 90% or at least 95% sequence identity. When less than the entire sequence is being compared for sequence identity, homologs and variants will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI website. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and non-human mammals.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

UGRP1: uteroglobin related protein 1. In one embodiment, the uteroglobin related protein 1 is a mammalian protein, such as a human protein or a murine protein. One example of a nucleic acid sequence encoding human UGRP1 is shown in SEQ ID NO:2. In another embodiment, the nucleic acid encodes a murine UGRP1 protein, such as a mouse, rat, or hamster UGRP1 protein. One specific, non-limiting example of a murine UGRP1 is the mouse UGRP1 shown in SEQ ID NO:3.

The human UGRP1 gene is about 2,900 base pairs in length and consists of three exons. The first intron of UGRP1 is about five to six-fold longer than the second intron, which resembles the structure of orthologous mouse UGRP1 gene. Also disclosed herein the nucleic acid sequence of the mouse UGRP1 gene.

UGRP1 promoter: An array of nucleic acid control sequences which direct transcription of a URGP1 nucleic acid in a host cell. One specific, non-limiting example of a UGRP1 promoter sequence is the human UGRP1 promoter sequence. An exemplary human UGRP1 promoter sequence is shown in SEQ ID NO:1. Another specific, non-limiting example of a UGRP1 promoter is the sequence shown in SEQ ID NO:1, wherein there is a G to A transition at position −112. Yet another specific, non-limiting example of the UGRP1 promoter sequence is the mouse UGRP1 promoter sequence. An exemplary mouse UGRP1 promoter sequence is shown in SEQ ID NO:19.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Unless otherwise explained, all technical and scientific terms used herein have the same meaning as commonly understood by one of ordinary skill in the art to which this invention belongs. The singular terms “a”, “an”, and “the” include plural referents unless context clearly indicates otherwise. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Sequences

SEQ ID NO:1 is the human UGRP1 promoter nucleic acid sequence.

SEQ ID NO:2 is a human UGRP1 nucleic acid sequence.

SEQ ID NO:3 is a mouse UGRP1 nucleic acid sequence.

SEQ ID NOS:4-10 are nucleic acid sequences of primers used to detect murine UGRP1 and uteroglobin/CCSP transcripts.

SEQ ID NO:11 is the nucleic acid sequence of a T7 primer.

SEQ ID NO:12 is the nucleic acid sequence of a UGRP1 specific primer.

SEQ ID NOS:13-16 are nucleic acid sequences of primers used to make a pGL3-190 mut 1 and mut 2, and −147 mut plasmids.

SEQ ID NOS:17-18 are nucleic acid sequences of primers used to generate mouse UGRP1-promoter specific probes.

SEQ ID NO:19 is the nucleic acid sequence of the mouse UGRP1 promoter.

SEQ ID NO:20 is the amino acid sequence of a portion of UGRP1 type A SEQ ID NO:21 is the amino acid sequence of a portion of mouse UGRP2.

SEQ ID NO:22 is the amino acid sequence of a portion of human UGRP 1.

SEQ ID NO:23 is the amino acid sequence of a portion of human UGRP2.

SEQ ID NOS:24 and 25 are primers that amplify the human UGRP1 promoter, but not the mouse UGRP1 transcript.

SEQ ID NO:26 is the amino acid sequence of a portion of mouse uteroglobin/CCSP.

SEQ ID NO:27 is the amino acid sequence of a portion of human mammaglobin A.

SEQ ID NO:28 is the amino acid sequence of a portion of rat prostatein C3.

### Promoter Sequences

Specifically disclosed herein is a polynucleotide sequence of mammalian UGRP1 promoters. In one specific, non-limiting example, the human UGRP1 promoter nucleotide sequence is SEQ ID NO:1 or a conservative variant thereof. In another specific, non-limiting example, the mouse UGRP1 promoter is SEQ ID NO:19 or a conservative variant thereof. An “isolated polynucleotide” is a polynucleotide that is not immediately contiguous with both of the coding sequences with which it is immediately contiguous (one on the 5′ end and one on the 3′ end) in the naturally occurring genome of the organism from which it is derived. Thus, in one embodiment, an isolated UGRP1 promoter from a specific species is not adjacent to the UGRP1 coding sequences from the same species. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences.

DNA sequences encoding a polypeptide can be expressed in vitro by DNA transfer into a suitable host cell. The host cell can be any cell in which a vector can be propagated and its DNA expressed. The cell may be prokaryotic or eukaryotic. Specific, non-limiting examples of a host cell include a cell from a cell line or a primary cell in culture. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

In the present invention, the UGRP1 sequences may be incorporated into an expression vector. In one embodiment, the expression vector is a plasmid, virus or other vehicle known in the art that has been manipulated by insertion or incorporation of the UGRP1 promoter sequences. A polynucleotide sequence which encodes any polypeptide of interest can be operatively linked to the UGRP1 promoter sequence. In one embodiment, the UGRP1 promoter sequence is operatively linked to a coding sequence; the UGRP1 promoter is ligated such that expression of the coding sequence is achieved under appropriate conditions. Thus the UGRP1 promoter sequence regulates the transcription of the nucleic acid sequence. Other expression control sequences such as, enhancers, transcription terminators, a start codon (i.e., ATG) in front of a protein-encoding gene, splicing signal for introns, maintenance of the correct reading frame of that gene to pen-nit proper translation of mRNA, and stop codons, can also be utilized in conjunction with the UGRP1 promoter sequence.

In one specific non-limiting example, an expression vector contains an origin of replication, a UGRP1 promoter, as well as a specific protein coding sequence of interest, and a sequence which allows phenotypic selection of the transformed cells. Protein coding sequences of interest include, but are not limited to, enzymes, receptors, antigenic epitopes, and markers. Vectors suitable for use in the present invention include, but are not limited to the T7-based expression vector for expression in bacteria (Rosenberg et al., Gene 56:125, 1987), the pMSXND expression vector for expression in mammalian cells (Lee and Nathans, J. Biol. Chem. 263:3521, 1988) and baculovirus-derived vectors for expression in insect cells.

The UGRP1 promoter can be utilized in eukaryotic cells. Hosts can include any mammalian cell. Methods of expressing DNA sequences having eukaryotic or viral sequences are well known in the art. Biologically functional viral and plasmid DNA vectors capable of expression and replication in a host are known in the art, and can be utilized with a UGRP1 promoter sequence.

Any host cell can be transformed with a vector including a UGRP1 promoter sequence. The genetic change is generally achieved by introduction of the DNA into the genome of the cell (i.e., stable). Transformation of a host cell with recombinant DNA may be carried out by conventional techniques as are well known to those skilled in the art. When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences including the UGRP1 promoter operably linked to a heterologous nucleic acid of interest, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982) from the UGRP1 promoter.

### Polymorphism Detection

A novel method is provided for detecting a respiratory disease or measuring the predisposition of a subject for developing a disease in the future by obtaining a biological sample from a subject; and screening the biological sample for the presence of a mutation in the UGRP1 promoter sequence. In one embodiment, the subject is a human but can also be any other organism, including, but not limited to, mammals such as a dog, cat, rabbit, cow, bird, rat, horse, pig, or monkey.

The biological sample may be any which is conveniently taken from the patient and contains sufficient information to yield reliable results. Typically, the biological sample will be a biological fluid or a tissue sample that contains, for example about 1 to about 10,000,000 cells. In one embodiment, the sample contains about 1000 to about 10,000,000 cells, or from about 1,000,000 to 10,000,000 somatic cells. It is possible to obtain samples which contain smaller numbers of cells (e.g. about 1 to about 1,000 cells) and then enrich the cells. In addition, with certain highly sensitive assays (e.g., RT-PCR) it is possible to get sample size down to single cell level. The sample need not contain any intact cells, so long as it contains sufficient biological material (e.g., nucleic acid, such as DNA or RNA; etc.) to assess the presence or absence of a mutation in the UGRP1 promoter in the subject.

The biological or tissue sample can be drawn from the tissue which is susceptible to the type of disease to which the detection test is directed. For example, the tissue may be obtained by surgery, biopsy, swab, or other collection method from the lung. In addition, a blood sample or a sputum sample can be used. In one embodiment, the biological sample is a blood sample. The blood sample may be obtained in any conventional way, such as finger prick or phlebotomy. Suitably, the blood sample is approximately 0.1 to 20 ml, or from about 1 to 15 ml, or about 10 ml of blood.

Screening for mutated nucleic acids can be accomplished by direct sequencing of nucleic acids. UGRP1 promoter nucleic acid may be sequenced to determine the exact nature of the mutation. Nucleic acid sequences can be determined through a number of different techniques which are well known to those skilled in the art. Nucleic acid sequencing can be performed by chemical or enzymatic methods. The enzymatic method relies on the ability of DNA polymerase to extend a primer, hybridized to the template to be sequenced, until a chain-terminating nucleotide is incorporated. The most common methods utilize didoexynucleotides. Primers may be labeled with radioactive or fluorescent labels. Various DNA polymerases are available including Klenow fragment, AMV reverse transcriptase, Thermus aquaticus DNA polymerase, and modified T7 polymerase. In one embodiment, in order to sequence the nucleic acid, sufficient copies of the material must first be amplified.

Southern hybridization is also an effective method of identifying differences in sequences. Hybridization conditions, such as salt concentration and temperature can be adjusted for the sequence to be screened. Southern blotting and hybridizations protocols are described in Current Protocols in Molecular Biology (Greene Publishing Associates and Wiley-Interscience, pages 2.9.1-2.9.10). Very high specific activity probe can be obtained using commercially available kits such as the Ready-To-Go DNA Labeling Beads (Pharmacia Biotech), following the manufacturer's protocol.

Restriction enzyme polymorphism is an additional method of identifying differences in sequences. Restriction enzyme polymorphism allows differences to be established by comparing the characteristic polymorphic patterns that are obtained when certain regions of genomic DNA are cut with various restriction enzymes. In one embodiment, the genomic DNA is amplified prior to being-cut with the restriction enzymes.

In one embodiment, the UGRP1 promoter sequence is amplified. Amplification of a selected, or target, UGRP1 promoter nucleic acid sequence may be carried out by any suitable means (Kwoh, D. and Kwoh, T., Am. Biotechnol Lab, 8, 14 ,1990). Examples of suitable amplification techniques include, but are not limited to, polymerase chain reaction, ligase chain reaction (see Barany, Proc Natl Acad Sci USA 88:189, 1991), strand displacement amplification (Walker, G. et al., Nucleic Acids Res. 20:1691, 1992; Walker. G. et al., Proc Natl Acad Sci USA 89:392, 1992), transcription-based amplification (see Kwoh, D. et al., Proc Natl Acad Sci USA , 86:1173, 1989), self-sustained sequence replication (or “3SR”) (see Guatelli, J. et al., Proc Natl Acad Sci USA, 87:1874 , 1990), the Q.beta. replicase system (see Lizardi, P. et al., Biotechnology, 6:1197, 1988), nucleic acid sequence-based amplification (or “NASBA”) (see Lewis, R., Genetic Engineering News, 12(9):1, 1992), the repair chain reaction (or “RCR”) (see Lewis, R., Genetic Engineering News, 12(9):1,1992), and boomerang DNA amplification (or “BDA”) (see Lewis, R., Genetic Engineering News, 12(9):1, 1992). In one specific non-limiting example, polymerase chain reaction is utilized.

Recently, single strand polymorphism assay (“SSPA”) analysis and the closely related heteroduplex analysis methods have come into use as effective methods for screening for single-base polymorphisms (Orita, M. et al., Proc Natl Acad Sci USA, 86:2766, 1989). In these methods, the mobility of PCR-amplified test DNA from clinical specimens is compared with the mobility of DNA amplified from normal sources by direct electrophoresis of samples in adjacent lanes of native polyacrylamide or other types of matrix gels. Single-base changes often alter the secondary structure of the molecule sufficiently to cause slight mobility differences between the normal and mutant PCR products after prolonged electrophoresis.

Ligase chain reaction is yet another recently developed method of screening for mutated nucleic acids. Ligase chain reaction (LCR) is also carried out in accordance with known techniques. LCR is especially useful to amplify, and thereby detect, single nucleotide differences between two DNA samples. In general, the reaction is called out with two pairs of oligonucleotide probes: one pair binds to one strand of the sequence to be detected; the other pair binds to the other strand of the sequence to be detected. The reaction is carried out by, first, denaturing (e.g., separating) the strands of the sequence to be detected, then reacting the strands with the two pairs of oligonucleotide probes in the presence of a heat stable ligase so that each pair of oligonucleotide probes hybridize to target DNA and, if there is perfect complementarity at their junction, adjacent probes are ligated together. The hybridized molecules are then separated under denaturation conditions. The process is cyclically repeated until the sequence has been amplified to the desired degree. Detection may then be carried out in a manner like that described above with respect to PCR.

In one embodiment, DNA amplification techniques such as the foregoing involve the use of a probe, a pair of probes, or two pairs of probes which specifically bind to non-mutated (native) UGRP1 promoter sequences, but do not bind to a mutated UGRP1 promoter, under the same hybridization conditions, and which serve as the primer or primers for the amplification reaction. Alternatively of a probe, a pair of probes, or two pairs of probes which specifically bind to a mutated UGRP1 promoter sequences, but do not bind to a non-mutated (native) UGRP1 promoter, under the same hybridization conditions, and which serve as the primer or primers for the amplification reaction.

Without further elaboration, it is believed that one skilled in the art can, using this description, utilize the present invention to its fullest extent. The following examples are illustrative only, and not limiting of the remainder of the disclosure in any way whatsoever.

### EXAMPLE 1

### T/EBP/NKX2.1

T/EBP (thyroid-specific enhancer-binding protein)/NKX2.1, also known as TTF1 (thyroid transcription factor 1), is a homeodomain-containing DNA-binding protein, which was originally characterized as a transcription factor regulating thyroid-specific expression of genes, including thyroglobulin (Civitareale et al., EMBO J 8:2537-2542,1989), thyroid peroxidase (Abrarnowicz et al., Eur J Biochem 203:467-473, 1992; Francis-Lang et al., Mol Cell Biol 12:576-588, 1992; Kikkawa et al., Mol Cell Biol 10:6216-6224,1990), TSH receptor (Civitareale et al., Mol Endocrinol 7:1589-1595, 1993; Shimura et al., Mol Endocrinol 8:1049-1069; 1994) and Na/I symporter (Endo et al., Mol Endocrinol 11:1747-1755, 1997) genes. T/EBP/NKX2.1 also controls transcription of genes specifically expressed in lung such as those encoding surfactant proteins(SP)-A (Bruno et al., J Biol Chem 270:6531-6536, 1995), B (Bohinski et al., Mol Cell Biol 14:5671-5681, 1994), and C (Kelly et al., J Biol Chem 271:6881-6888, 1996), and uteroglobin/Clara cell secretory protein (CCSP) (Ray et al., Mol Cell Biol 16:2056-2064, 1996; Sawaya et al., Mol Cell Biol 13:3860-3871, 1993). The tissue specific pattern of expression of the genes is believed to be conferred by unique combination of T/EBP/NKX2.1 with other transcription factors, which includes PAX8 and TTF2 in the thyroid (Francis-Lang et al., Mol Cell Biol 12:576-588, 1992; Damante et al., Biochim Biophys Acta 1218:255-266, 1994), and hepatocyte nuclear factor (HNF)3s and the HNF3/forkhead homologs (HFHs) in the lung (Bohinski et al., Mol Cell Biol 14:5671-5681, 1994; Clevidence et al., Dev Biol 166:195-209, 1994). T/EBP/NKX2.1 is expressed in lung, thyroid, and a part of brain in adult and during embryogenesis (Guazzi et al., EMBO J 9:3631-3639, 1990; Lazzaro et al., Development 113:1093-1104, 1991; Mizuno et al., Mol Cell Biol 11:4927-4933, 1991), the latter of which suggested the involvement of T/EBP/NKX2.1 in development. In fact, suppression of T/EBP/NKX2.1 by antisense oligonucleotides in vitro using lung organ cultures abrogated normal branching morphogenesis (Minoo et al., Dev Biol 172:694-698, 1995). Further, targeted disruption of the T/ebp/Nkx2.1 locus resulted in immediate postnatal death due to respiratory failure caused by profoundly hypoplastic lungs (Kimura et al., Genes Dev 10:60-69, 1996). In addition to the lung, these mice lack the thyroid and pituitary, and exhibit severe defects in the ventral forebrain such as hypothalamus and basal ganglia (Kimura et al., Genes Dev 10:60-69, 1996; Minoo et al., Dev Biol 209:60-71, 1999; Yuan et al., Dev Dyn 217:180-190 22, 1999; Sussel et al., Development 126:3359-3370; 1999; Takuma et al., Development 125:4835-4843, 1998). Thus, T/EBP/NKX2.1 appears to serve as one of the master regulatory genes responsible for organogenesis of the thyroid, lung, and ventral forebrain. However, the exact impact of the developmental block, resulting from inactivation of the T/ebp Nkx2.1 locus on structural morphogenesis and differentiation of the cells in these organs remains unclear.

In the lung, T/EBP/NKX2.1 is expressed in all epithelial cells early in pulmonary morphogenesis, but the expression becomes progressively restricted to alveolar type II and Clara cells (Yuan et al., Dev Dyn 217:180-190 22, 1999). Analyses of the T/ebp/Nkx2.1-null mouse suggested that T/EBP/NKX2.1 may function in the establishment of pattern formation and pulmonary morphogenesis during early embryonic development. The lack of T/EBP/NKX2.1 expression leads to the condition called tracheocsophageal fistula, where the trachea and esophagus share a common tube (Minoo et al., Dev Biol 209:60-71, 1999). The main stem bronchi bifurcate from this common structure, connecting to severely hypoplastic lungs. These phenotypes found in T/ebp/Nkx2.1-null mouse must be related to the ability of T/EBP/NKX2.1 to activate and/or suppress specific downstream target genes. One such category of target genes consists of SP-A, B, and C, and uteroglobin/CCSP in lung, all of which are not expressed in T/ebp/Nkx2.1-null embryo lungs (Minoo et al., Dev Biol 209:60-71, 1999). These genes are however, not known to have morphoregulatory function. In T/ebp/Nko2.1-null embryo lungs, expression of some extracellular matrix proteins and their cellular receptors including collagen type IV and a integrins, and some growth factors such as Vegf3 and Bmp4 are reduced or absent (Minoo et al., Dev Biol 209:60-71, 1999; Yuan et al., Dev Dyn 217:180-190 22, 1999). Whether the abnormal phenotype in T/ebp/Nkx2.1-null embryo lungs is entirely or partially due to reduction or absence of expression of these genes remains to be examined. Thus, a potential T/ebp/Nkx2.1 target gene, mouse UGRP1 that encodes a uteroglobin/CCSP-related protein was cloned. The following methods were used in the murine studies.

Identification of Lung-specific Gene UGRP1 by Suppressive Subtractive Hybridization

T/ebp/Nkx2.1 (+/−) mice were bred to generate T/ebp/Nkx2.1-null embryos (Kimura et al., Genes Dev 10:60-69, 1996). Embryos were obtained by dissection of pregnant mice at E16.5 and genotyping was performed by PCR using yolk sacs. Noon on the day when the vaginal plug was detected was considered as stage E0.5. Total RNA was isolated from lungs of null mutant embryos (driver) and wild type embryos (tester) using ULTRASPEC™ RNA Isolation System (Biotecx Laboratories, Houston, Tex.), and was used as template to synthesize double-stranded cDNAs using a SMART™ PCR cDNA Synthesis kit (Clontech Laboratories, Palo Alto, Calif.). Suppressive subtractive hybridization (SSH) and differential screening were performed using PCR-Select-™ cDNA Subtraction Kit (Clontech) and PCR-Select Differential Screening kit (Clontech), respectively according to the manufacture's instructions. Clones that hybridized with only the forward-subtracted probe were selected for virtual Northern blot analyses, which uses cDNAs instead of RNAs as a source of expressed genes. The membrane containing cDNAs synthesized by SMART™ PCR cDNA Synthesis kit was prehybridized at 60° C. in ExpressHyb™ hybridization solution (Clontech) for 30 min and hybridized in fresh buffer with denatured random primer-labeled probe at 60° C. for 3 h. After hybridization, the blot was washed twice in 2×SSC (2×SSC is 0.3 M NaCl and 30 mM Na Citrate, pH 7.0) containing 0.1% SDS at room temperature for 10 min, followed by once with 0.1×SSC containing 0.1% SDS at 50° C. for 20 min. The filter was then exposed to a PhosphoImager screen overnight. Signal intensities were analyzed using ImageQuant program (Molecular Dynamics, Inc., Sunnyvale, Calif.). Differentially expressed clones were subjected to DNA sequencing analyses.

Cloning and DNA Sequencing

Adult mouse lung CDNA library in the λZAPII vector (Stratagene, La Jolla, Calif.) was screened by plaque hybridization using CDNA isolated from SSH as a probe. Hybridization was carried out at 65° C. in 6×SSC, 0.5% SDS, 5× Denhardt's, 0.1 mg/ml of denatured salmon sperm DNA for 16 h. The membrane was washed twice with 2×SSC containing 0.1% SDS at room temperature for 10 min and once with 0.1×SSC containing 0.1% SDS at 55° C. for 30 min. Positive plaques were picked from plates and were subjected to secondary and tertiary screenings. The UGRP1 genomic DNA was isolated from a mouse BAC genomic library (Incyte Genomics, St. Louis, Mo.) using labeled UGRP1 cDNA as probe.

The cDNAs encoding mouse UGRP2 and human UGRP1 and 2 were isolated by RT-PCR using total RNAs prepared from adult mouse and human lungs (Ambion, Austin, Tex.), respectively and primers designed based on EST sequences that exhibited similarities to the mouse UGRP1 cDNA sequence. In the case of mouse UGRP2 cDNA, a mouse lung cDNA library was also screened using a fragment obtained by RT-PCR as probe. The identity of both cDNA clones obtained by RT-PCR and library screening was confirmed by sequencing. Sequencing was performed using an ABI prism dye terminator cycle sequencing ready reaction kit and a model 377 DNA sequencer (PE Applied Biosystems, Foster City, Calif.).

The nucleotide sequences reported in this paper appear in the GenBank databases under the following accession numbers; UGRP1 type A mRNA: AF274959 (SEQ ID NO:3), type B mRNA: AF274960, type C mRNA: AF274961, mUGRP2: AF313456, EST AI391046, hUGRP1: AF313455 (SEQ ID NO:2), EST AI355612, EST AI355302, hUGRP2: AF313458, EST AW974727, all of which are herein incorporated by reference.

Determination of the Transcription Start Site

The transcription start site of the mouse UGRP1 gene was determined by SMART™ RACE cDNA amplification kit (Clontech) using adult mouse lung total RNA. DNA sequence analyses' indicated the presence of multiple transcription start sites. Since the most clones (eight out of sixteen) had the exact sequence (91 bp upstream from ATG), we refer to this site as the major transcription start site.

Chromosomal Mapping

A UGRP1 probe of 11 kb genomic DNA labeled with biotin or digoxigenin was used for in situ hybridization of chromosomes derived from mouse spleen cultures. Conditions of hybridization, detection of hybridization signals, digital-image acquisition, processing and analysis, direct fluorescent signal localization on banded chromosomes were performed as previously described (Zimonjic et al., Cancer Genet Cylogenet 80:100-102, 1995). To confirm the identity of chromosomes, preparations were rehybridized with mouse chromosome 18 painting probe and previously observed labeled metaphases were recorded.

RNA Analyses

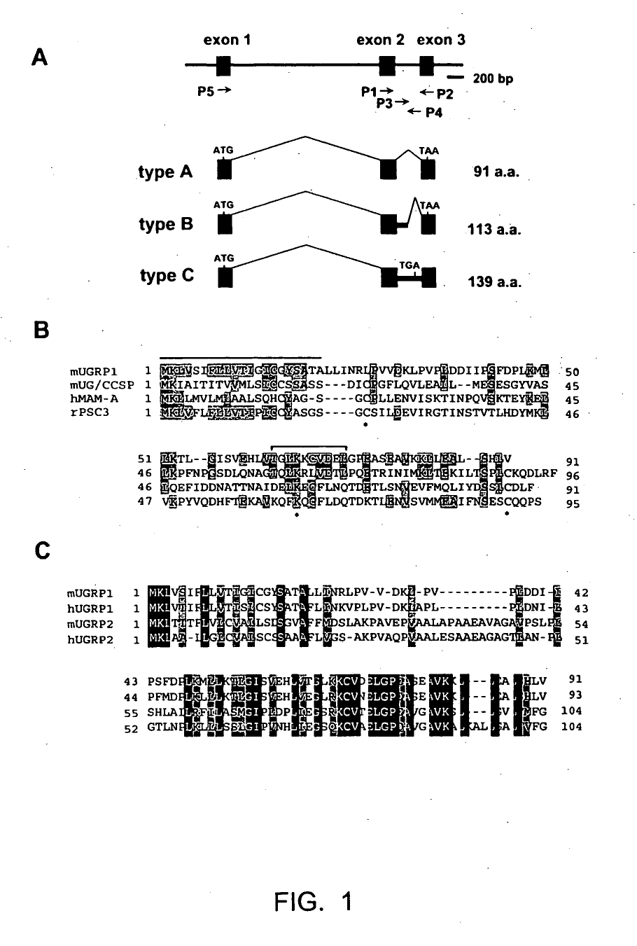

Reverse transcription of mRNAs was carried out in a final volume of 20 μl containing 2 μg of total RNA, 4 μl of 5× first strand synthesis buffer (Life Technologies), 1 μl of a mixture of four dNTPs (2.5 mM each), 2 μl of 0.1 M dithiothreitol (DTT) and 100 ng of random primers. After incubation at 37° C. for 2 min, 200 units of Super Script II reverse transcriptase (Life Technologies) was added and the incubation continued for 60 min at 37° C. Single stranded cDNAs in 0.1 μl of the reaction mixture were amplified by PCR using AmpliTaq DNA polymerase (PE Applied Biosystems) under the following conditions; denaturation at 94° C. for 30 s, annealing at 60° C. for 30 s, and extension at 72° C. for 1 min, for 30 or 25 cycles when total RNAs or plasmids were used as template, respectively. The oligonucleotide primers used to detect UGRP1 and uteroglobin/CCSP transcripts were as follows (see FIG. 1A for UGRP1):

| | | |
|---|---|---|
| P1: | 5′- | |
| | GTAGAACATCTGGTGACAGG-3′, | (SEQ ID NO: 4) |
| P2: | 5′- | |
| | CAGCCAGAGTGAGCAAATCC-3′, | (SEQ ID NO: 5) |
| P3: | 5′- | |
| | TCCCTGGGAGAAGCCTTTGC-3′, | (SEQ ID NO: 6) |
| P4: | 5′- | |
| | GGAGTCCCTGGGATATGCAC-3′, | (SEQ ID NO: 7) |
| P5: | 5′- | |
| | GACTGCATTCCAAAGTCCCG-3′, | (SEQ ID NO: 8) |
| uteroglo- | 5′- | |
| bin/CCSP | CTACAGACACCAAAGCCTCC-3′, | (SEQ ID NO: 9) |
| forward: | | |
| uteroglo- | 5′- | |
| bin/CCSP | AAGGAGGGGTTCGAGGAGAC-3′. | (SEQ ID NO: 10) |
| reverse: | | (33) |

Northern blotting was carried out using a multiple mouse tissue northern blot (Clontech) or total RNAs isolated from adult mouse lung and thyroid. The blots were hybridized with a full-length UGRP1 cDNA as a probe. Hybridization was performed in ExpressHyb™ Hybridization Solution at 68° C. for 2 h. The membrane was washed twice with 2×SSC containing 0.1% SDS at room temperature for 10 min and twice with 0.1×SSC containing 0.1% SDS at 55° C. for 20 min, followed by exposure to X-ray film at −80° C. For the analyses of UGRP1, UGRP2, and uteroglobin/CCSP expression levels in uterus, mice were daily intraperitoneally injected with progesterone (3 mg/kg) in phosphate buffered saline (PBS) or PBS alone for four days, and RNA was prepared on the 5th day.

Luciferase Plasmid Construction and Site-directed Mutagenesis

A 9-kb BglII fragment containing 0.9 kb of the 5′-flanking sequence of mouse UGRP1 genomic DNA was subcloned into the BamHI site of pBluescript II, and PCR was performed with T7 primer (5′-GTAATACGACTCACTATAGGGC-3′, SEQ ID NO:11) and a UGRP1 gene-specific primer (5′-TGCCTGTGATGTTTTCCGGG-3+; +85 to +66, SEQ ID NO:12). The PCR product was subcloned into pCR2.1 (Invitrogen, Carlsbad, Calif.), and an XbaI-BamHI fragment from this plasmid was inserted into the NheI-BglII site of the pGL3-Basic luciferase reporter vector (Promega, Madison, Wis.) to generate the pGL3-907. plasmid. This construct was further digested with KpnI and MluI for preparation of deletion plasmids-using Exonuclease III (New England Biolabs, Beverly, Mass.) and S1 nuclease (Life Technologies). Six deletion constructs (pGL3- 18, −67, −147, −190, −242, and −907) were sequenced to determine the exact sequences.

Site-directed mutagenesis of potential T/EBP/NKX2.1 binding site was introduced into the pGL3-190 and −147 plasmids by using QuikChange™ Site-Directed Mutagenesis kit (Stratagene). The following primers were used to make a pGL3-190 mut 1 and mut 2, and −147 mut plasmids:

mut 1: 5′-
GGTGCCAGAACATTTCTCTACGGGAGACTACTTCTGTG-
3′
(SEQ ID NO: 13)
and
5′-
CACAGAAGTAGTCTCCCGTAGAGAAATGTTCTGGCACC-3′
(complementary strand,,
SEQ ID NO: 14)
mut 2: 5′-
GTGGAAAACCCTTCCTAATGTTTAGTTAGGAAGATTG-
CCCTG-3′
(SEQ ID NO: 15)
and
5′-
CAGGGCAATCTTCCTAACTAAACATTAGGAAGGGTTTTCCAC-3′
(complementary strand,.
SEQ ID NO: 16).

Transfeclion and Reporter Gene Assays

The human lung adenocarcinoma cell line NCI-H441 was maintained in RPMI 1640 medium containing 10% fetal calf serum. HeLa cells were cultured in minimum essential medium containing 10% fetal calf serum. Cells in 12 well plates at 50-70% confluency were transfected by using Effectene transfection reagent (Qiagen, Valencia, Calif.) with 250 ng of reporter plasmid, 25 ng of expression vector and 25 ng of pCH110 (Amersham Pharmacia Biotech) as an internal control. After 48 h, the cells were harvested in Reporter lysis buffer (Promega), and the lysates were assayed for β-galactosidase and luciferase activities using High Sensitivity β-Galactosidase Assay Kit (Stratagene) and Luciferase Assay System (Promega), respectively. To correct for transfection efficiency, luciferase activity was normalized to β-galactosidase activity. Relative luciferase activity of various mouse UGRP1 promoter constructs was expressed based on the activity of pGL3-Basic in the presence of the same trans-activating plasmid as 1. Data are the mean value of at least three experiments (duplicate samples)±S.D.

DNase I Footprinting

A 5′-end-labeled probe of the 307-bp mouse UGRP1 promoter region was generated by PCR using pGL3-907 as a template and a sense primer; 5′-AAAGGATCCTATAGGAAAGCATTCCTCTC-3′ (SEQ ID NO:17), and an antisense primer; 5′-AAACTCGAGTGATGGCTGCTTTTCCTCAG-3′ (SEQ ID NO:18). Recombinant T/EBP/NKX2.1 protein was produced according to the manufacture's instruction (Novagen, Madison, Wis.) by using the pET-30a (+)-T/EBP/NKX2.1 expression vector (kindly provided by Dr. Leonard Kohn, Ohio University, Athens, Ohio). The DNase I footprinting reaction was performed using a SureTrack Footprinting Kit (Amersham Pharmacia Biotech). Briefly, recombinant T/EBP/NKX2.1 protein (2 μg), or BSA(30 μg) for a naked DNA control, was incubated with 20,000 cpm of probe for 30 min, and were subjected to DNase I digestion for 1 min at room temperature. The DNA fragments were separated on 6% polyacrylamide, 7M urea sequencing gels using the dideoxy sequencing reaction product (fmol DNA Sequencing System; Promega) as a size marker.

Electrophoretic Mobility Shift Assays

Nuclear extracts of NCI-H441 cells were prepared as described (Dignam et al., Nucleic Acids Res 11:1475-1489, 1983). Nuclear extracts (15 μg) and, when indicated, unlabeled oligonucleotide competitor DNAs were preincubated in 23 μl of gel mobility shift assay buffer (10 mM HEPES-KOH (pH 7.9), 50 mM KCl, 0.6 mM EDTA, 5 mM MgCl₂, 10% glycerol, 5 mM DTT, 0.7 mM PMSF, 2 μg/μl pepstatin A, 2 μg/μl leupeptin, and 87 μg/μl poly (dI-dC) (Amersham Pharmacia Biotech)) for 10 min on ice.

Oligonucleotide probe (1×10₅ cpm) was added to the mixture, and the mixture was incubated for an additional 30 min at room temperature. For antibody supershift analyses, 1 μl of anti-TTF-1 monoclonal antibody (Lab Vision Corporation, Fremont, Calif.) was added and the incubation was continued for an additional 1 h. Protein-DNA complexes were separated from free probe by 5% non-denaturing polyacrylamide gel electrophoresis. After electrophoresis, the gel was blotted onto Whatman No. 3MM paper, dried, and exposed to X-ray film.

Western Blot Analysis

Three forms of cDNAs encoding type A, B and C proteins were amplified by PCR and inserted into EcoRI and XhoI site of pcDNA3.1/Myc-His(+) A vector (Invitrogen). Transient transfection into COS-1 cells was performed using Effectene transfection reagent (Qiagen). After 2 days, cells and conditioned media were collected, separated on 13% SDS-polyacrylamide gels under reducing and non-reducing conditions, and electrophoretically transferred to nitrocellulose membrane (Schleicher & Schuell). The filter was incubated in PBS containing 5% skim milk, and then for 1 h with 250-fold diluted c-myc 9E10 polyclonal antibody (Santa Cruz Biotechnology, Santa Cruz, Calif.). The filter was washed in PBS containing 0.1% Tween 20, incubated with horseradish peroxidase (HRP)-conjugated anti-rabbit IgG (Amersham Pharmacia Biotech), and then washed with the same buffer. Protein bands were detected using ECL Western blotting detection reagent (Amersham Pharmacia Biotech).

Immunohistochemistry

A cDNA segment encoding the mature 70 amino acids of UGRP1 type A polypeptides was prepared by PCR with the use of full-length UGRP1 as a template, and was subcloned into a bacterial expression vector pET32a(+) (Calbiochem-Novabiochem Corp., La Jolla, Calif.), placing the UGRP1 sequence in-frame downstream of a hexahistidine tag. The tagged UGRP1 peptide was expressed in E. coli BL21(DE3) by induction with 1 mM isopropylthio-β-galactoside for 5 hr. Cells were collected and lysed in native conditions, and tagged peptide was purified on a nickel-NTA agarose column, followed by SDS-polyacrylamide gel. The purified peptide was used to prepare UGRP1 antibody in rabbits (Macromolecular Resources, Fort Collins, Colo.). The anti-mouse uteroglobin/CCSP antibody was a kind gift of Dr. Anil Mukheriee (NICHD, Bethesda, Md.). Immunohistochemistry was carried out using 2000 and 1000-fold dilution of UGRP1 and uteroglobin/CCSP antibodies, respectively and Vectastain ABC Rabbit Elite Kit (Vector Laboratories, Burlingame, Calif.).

Mouse Sensitization

Female BALB/cJ mice (Jackson Labs, Bar Harbor, Me.), 6-7 weeks old, were used in these studies. They were housed in a controlled environment with a 12-hr light on/12-hr light off cycle and had access to food and water ad lib. The animals were treated in accord with PHS guidelines and under a protocol approved by Genaera Corp. Institutional Animal Care and Use Committee. Mice were sensitized with intraperitoneal administrations of a mixture of Aspergillus fumigatus extract (Bayer, Elkhart, Ind.; 200 μg/mouse) and alum (Imject®, Pierce Chemicals, Rockford, Ill.; 2.25 mg/mouse) on study days 0 and 14 and subsequent intranasal administrations of 25 μL of Aspergillus fumigatus extract (final concentration 1:50 w/v in 10% glycerol) while under light inhaled anesthesia on study days 24, 25, and 26. Mice that were not sensitized, nor treated, were designated “naive.”

Sensitized mice were treated intraperitoneally with either: 1) dexamethasone-21-phosphate (Sigma, St. Louis, Mo.) at a dose of 2.5 mg/kg twice per week for a total of nine administrations (Af+Dex) or 2) saline (0.9% sodium chloride injectable, USP, Baxter, Co. Deerfield, Ill.) as vehicle control (Af). Mice were also treated with dexamethasone-21-phosphate alone in the same schedule. On study day 28, mice were euthanized, and lung tissues were harvested (n=1-2/group) and immediately frozen in liquid nitrogen for later analyses of mRNA.

### EXAMPLE 2

### Isolation and Characterization of T/ebp/Nkx2.1 Downstream Target Gene

To isolate putative T/EBP/NKX2.1 downstream target genes, a suppressive subtractive hybridization method was used to generate a cDNA library of clones that were enriched in the lungs of E16.5 wild-type embryos versus T/ebp/Nkx2.1-null embryos. The latter lung is severely hypoplastic and does not present any characteristics beyond proximal lung morphogenesis (Yuan et al., Dev Dyn 217:180-190 22, 2000). It is therefore possible that the suppressive subtractive cDNA library we used could represent different population of cells between normally differentiated and developmentally arrested embryonic lungs rather than T/EBP/NKX2.1-regulated genes. One hundred and ninety-two clones were initially picked which were then probed with forward-subtracted cDNAs (wild-type) or the reverse-subtracted cDNAs (mutant). Twenty-seven clones that gave stronger signals with the forward-subtracted probe than the reverse probe were subjected to virtual northern blotting analyses. Five clones were found to be differentially expressed. Sequence analyses revealed that one of the clones encodes a polypeptide exhibiting sequence similarity to the uteroglobin/CCSP family of proteins (Mukherjee et al., Cell Mol Life Sci 55:771-787, 1999; Watson et al., Cancer Res 56:860-865, 1996; Margraf et al., Am J Respir Cell Mol Biol 9:231-238, 1993; Parker et al., J Biol Chem 258: 12-15, 1983). Therefore, this gene was referred to as UGRP1 (which encodes the protein UGRP1, uteroglobin-related protein 1).

To isolate a full-length UGRP1 cDNA, a mouse adult lung CDNA library was screened, and eight clones with a positive hybridization signal were identified in 1×10⁶ recombinant phage. After cloning and sequencing, three appeared to contain a full-length cDNA. Two additional cDNAs were isolated by RT-PCR that differ at their C-terminal sequences. These were used to classify the transcripts into three types A, B and C, with type A being the full-length cDNA obtained through the library screening. The three polypeptides A, B and C consist of 91, 113 and 139 amino acids, respectively (FIG. 1A). Complementary DNAs for type B and C transcripts were not found by cDNA library screening, suggesting that these two transcripts may be rare. Computer analyses revealed that the first 21 residues of the UGRP1 polypeptide may function as a signal sequence for targeting the protein to a secretory pathway (FIG. 1B).

A BLAST search of the type A amino acid sequence for similar proteins exhibited similarities to uteroglobin/CCSP family of proteins. Mouse UGRP1 has overall amino acid sequence identity of 25, 18 and 27% to mouse uteroglobin/CCSP (Margraf et al., Am J Respir Cell Mol Biol 9:231-238, 1993), human mammaglobin A (Watson et al., Cancer Res 56:860-865, 1996), and rat prostatein C3 (Parker et al., J Biol Chem 258:12-15, 1983), respectively (FIG. 1B). Significant similarity was found in the signal sequence at the N-terminus and amino acid residues 63 through 72, the area called antiflammin that is believed to be responsible for phospholipase A ₂-inhibitory activity of the uteroglobin/CCSP (Mukherjee et al., Cell Mol Life Sci 55:771-787, 1999). UGRP1 signal sequence exhibits particularly high similarity to that of rat prostatein C3.

Several mouse and human EST sequences were also identified that demonstrate similarities to the mouse UGRP1. Using RT-PCR with a part of EST sequences as primers, mouse homologous gene, UGRP2 and human orthologous genes to each mouse gene, UGRP1 and 2 were obtained. The human UGRP1 and 2, and mouse UGRP2 show 81, 41, and 33% amino acid sequence identity to mouse UGRP1, respectively (FIG. 1C), which suggests that they consist of a new gene family.

### EXAMPLE3

UGRP1 Genomic Structure, Alternatively Spliced Transcripts, and Chromosomal Location

In order to analyze UGRP1 genomic structure and to define the origin of the three types of transcripts, a mouse BAC genomic library was screened using the full-length type A cDNA as a probe. The mouse UGRP1 gene is composed of three exons and two introns when compared with the most abundant type A cDNA sequence. All the exon/intron boundaries match the consensus sequence for RNA splicing. Interestingly, the whole or N-terminal half of intron 2 can be alternatively retained in the transcripts, which appears to be responsible for the production of type B and C transcripts (FIG. 1A). Alternatively, they could represent incompletely spliced RNA transcripts. In the type B transcript, the N-terminal half of intron 2 encodes additional 22 amino acids that are inserted at residue 85 of the type A polypeptide. In the type C transcript, the complete intron 2 sequence is retained, which results in 33 unique amino acids at its C-terminus after residue 85 due to a stop codon present in the intron sequence.

Mouse chromosome spreads that were hybridized with biotin or digoxigenin-labeled genomic probes had specific fluorescent signals at identical sites on both chromosomes 18 in 40 out of 50 metaphases randomly selected for recordings. This was the only site with a double symmetrical fluorescent signal. Occasionally, single randomly distributed fluorescent spots were observed. Twenty five metaphases without overlapping chromosomes were analyzed by imaging of DAPI-enhanced G-like banding. Symmetrical fluorescence signal was localized at region 18C-D where we assign the location of UGRP1 gene. This region is homologous with human chromosome 5q31-q34 (DeBry et al., Genomics 33:337-351, 1996; Searle et al., Ann Hum Genet 53:89-140, 1989): multiple disorders such as cortisol resistance, refractory macrocytic anemia, 5q syndrome, and Treacher Colins mandibulofacial dysostosis are located in this region (McKusick et al., J Med Genet 30:1-26, 1993). Translocations specific for acute lymphoblastic leukemia are also localized to the region (Mitelman et al., Report of the committee on chromosome changes in neoplasia. The Johns Hopkins University Press, Baltimore-London, 1995). This region is further known to contain at least one asthma susceptibility locus (A genome-wide search for asthma susceptibility loci in ethnically diverse populations. The Collaborative Study on the Genetics of Asthma (CSGA). Nat Genet 15:389-392, 1997; Cookson et al., Hum Mol Genet 9:2359-2364, 2000; Postma et al., N Engl J Med 333:894-900, 1995).

### EXAMPLE 4

### Expression of UGRP1

Using type A cDNA as a probe, UGRP1 expression was examined in adult mouse tissues by northern blotting analyses. A single 0.5 kb transcript corresponding to type A was clearly detected in the lung. Since T/EBP/NKX2.1 is expressed in the thyroid, this tissue was also examined for UGRP1 expression. Longer exposure did reveal expression in the thyroid. No clear signal was found for type B and C transcripts that would cross-hybridize to the probe, further suggesting that type B and C transcripts are expressed at low levels. Since uteroglobin/CCSP was originally identified that is expressed in pregnant rabbit uterus and is induced by progesterone (Mukherjee et al., Cell Mol Life Sci 55:771-787, 1999; Mukherjee et al., Ann NY Acad Sci 923, 2000), mouse uterus with and without progesterone treatment was examined for the expression of UGRP1, UGRP2 and uteroglobin/CCSP. RT-PCR analysis did not detect any transcripts in either case. Thus, UGRP1, UGRP2 and uteroglobin/CCSP mRNAs are neither expressed nor induced by progesterone in mouse uterus, at least under the conditions used.

UGRP1 expression was also examined using RT-PCR with exon 1 (P5) and 3 (P2)-specific primer pair on E12.5 and 16.5 embryonic lung mRNAs obtained from wild-type and T/ebp/Nkx2.1-null mouse (FIG. 1A). In wild-type embryo lungs, a band corresponding to type A transcript (380 bp) was barely detected at E12.5, but became intense by E16.5. In contrast, markedly reduced expression was noted in E16.5 T/ebp/Nkx2.1-null embryo lungs. This may reflect the absence of an inductive effect of T/EBP/NKX2.1 and/or the absence of differentiated cells that normally express the gene.

The presence of other types of transcripts was confirmed by RT-PCR using various combinations of primers and E18.5 wild-type embryo lung mRNAs as template, or the individual A, B and C CDNA clone as a control template. In embryo lungs, a fragment corresponding to type A (167 bp), and both type B (130 bp) and C (394 bp) transcripts were demonstrated using exon 2 (P1) and 3 (P2)-specific, and intron 2 5′-region (P3) and exon 3 (P2)-specific primer pairs, respectively. A faint, but clear band corresponding to type C transcript (410 bp) was exhibited by using exon 1 (P5) and intron 2 3′-region (P4)-specific primer pairs. Although the signal from RT-PCR is not necessarily proportional to the expression level, these data again support the finding that type A transcript is most abundant.

### EXAMPLE 5

### UGRP1 Promoter is Trans-activated by T/EBP/NKX2.1

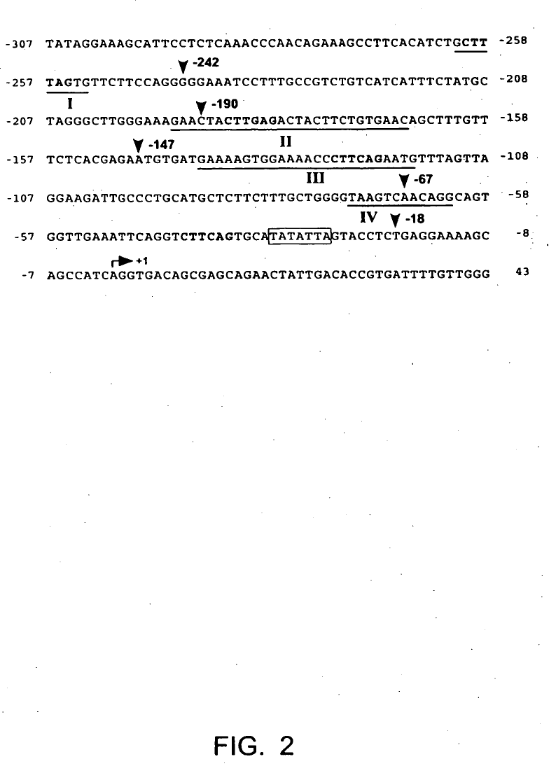

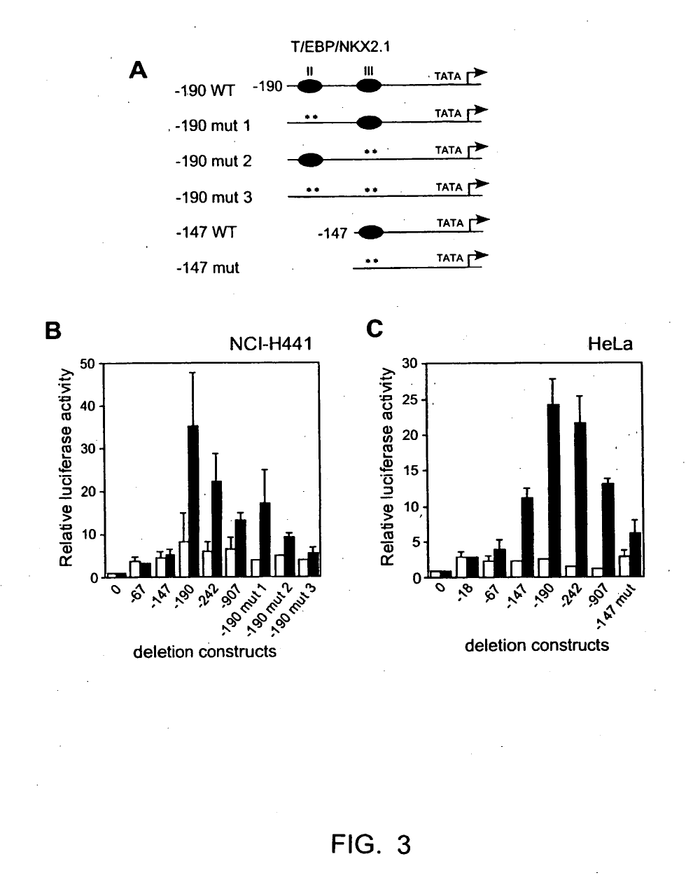

In order to demonstrate that UGRP1 promoter sequences are responsive to activation by T/EBP/NKX2.1, a DNA fragment containing the 5′-flanking region of the mouse UGRP1 gene was isolated and sequenced (FIG. 2, SEQ ID NO:19). A major transcription initiation site was determined by the 5′ RACE method using adult mouse lung mRNA as a template. A TATA box is located at position −26 bp. Four minimum consensus sequences for a possible T/EBP/NKX2.1 binding site (CTNNAG) (Bohinski et al., Mol Cell Biol 14:5671-5681, 1994) were identified at positions −255, −182, −120 and −37 bp within 307 base pairs of the upstream sequences. Six UGRP1 promoter-luciferase constructs (pGL3-18, −67, −147, −190, −242, and −907) were used to map regions responsible for UGRP1 transcriptional activity by co-transfecting into NCI-H441 or HeLa cells with either a pCMV4-T/EBP/NKX2.1 expression plasmid or a control pCMV4 vector (FIG. 3). NCI-H441 cells endogenously express T/EBP/NKX2.1 whereas HeLa cells do not. In NCI-H441 cells, a construct containing from +72 to −147 bp of the 5′ flanking sequence (pGL3-147) showed similar luciferase activity with and without co-transfection of expression plasmid. Construct −190 demonstrated approximately twice the activity as the construct −147 when control pCMV4 vector was present. Co-transfection of T/EBP/NKX2.1 expression plasmid further increased the activity approximately four fold. This increase of activity by co-transfection of the expression plasmid was probably due to insufficient amount of endogenous T/EBP/NKX2.1 present in NCI-H441 cells for full activity. A similar phenomenon was previously reported (Oguchi et al., Endocrinology 139:1999-2006, 1998). In the case of HeLa cells, constructs −147 and −190 exhibited approximately five fold and further two fold increase in luciferase activity, respectively by co-transfection of T/EBP/NKX2.1 expression plasmid as compared with the control vector alone. Such activity increase was not observed with the −67 construct. These results indicate that T/EBP/NKX2.1 binding elements necessary to activate UGRP1 gene transcription may be located between −190 and −147, and −147 and −67 bp. The nucleotide sequence in this region contains two consensus T/EBP/NKX2.1 binding sites (FIG. 2).

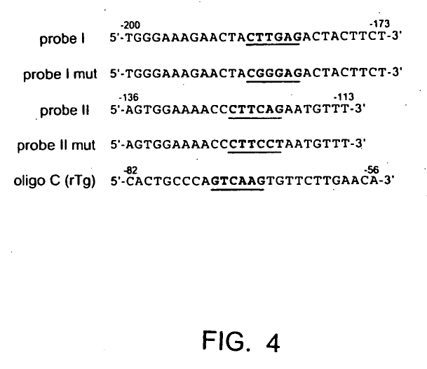

In order to more precisely localize the T/EBP/NKX2.1 binding sites between −67 and −190 bp, DNase I footprinting analyses were carried out using bacterially-expressed recombinant T/EBP/NKX2.1 and the 307-bp mouse UGRP1 gene promoter sequence (FIG. 2). Four protected regions were obtained; the region II and III each contained a consensus T/EBP/NKX2.1 binding sites at −182 and −120 bp, respectively. Since the protected region I is located upstream of the −242 construct, this region was not examined further. Electromobility shift assays were then performed with nuclear extracts from NCI-H441 cells to define the function of possible T/EBP/NKX2.1 binding elements at −182 and −120 bp (FIG. 4). Oligonucleotides, Probe 1 (−200 to −173 bp) and Probe 11 (−136 to −113 bp) (FIG. 4), each containing T/EBP/NKX2.1 consensus binding site (CTNNAG) (Bohinski et al., Mol Cell Biol 14:5671-5681, 1994) formed a specific protein-DNA complex, which was inhibited by the addition of 100 and 500-fold, and 100-fold excess of unlabeled specific oligonucleotide, respectively, but not by non-specific oligonucleotide. Oligonucleotides, each containing a respective mutated T/EBP/NKX2.1 binding site (Probe I mut and Probe II mut, FIG. 4) did not compete for complex formation whereas oligonucleotide containing the T/EBP/NKX2.1 binding site identified in the rat thyroglobulin gene (Civitareale et al., EMBO J 8:2537-2542, 1989) did compete. Anti-T/EBP/NKX2.1 monoclonal antibody produced a faint, but clear supershifted band. The third T/EBP/NKX2.1 consensus binding site found at −37 bp did not produce any specific protein-DNA complex, indicating that T/EBP/NKX2.1 does not bind to this site. Interestingly, when the DNase I footprinting protected area IV was used in gel shift analyses using NCI-H441 nuclear extracts, no specific protein-DNA complex was obtained. Thus, each one of the two T/EBP/NKX2.1 consensus binding sites appears to be responsible for the increase of luciferase activity with constructs −147 and −190, respectively.

In order to confirm the functional relationship between these two T/EBP/NKX2.1 consensus binding sites and UGRP1 gene transcriptional activity, construct −190 with mutated T/EBP/NKX2.1 binding sites (FIGS. 2 and 4) were used for transfection analyses in NCI-H441 cells (FIGS. 3B and 4). The luciferase activity in the presence of T/EBP/NKX2.1 expression vector decreased approximately half as compared with the construct −190 when the distal T/EBP/NKX2.1 binding site to the transcriptional start site was mutated (−190 mut 1) (FIG. 3). Construct −190 mut 2 that has the proximal T/EBP/NKX2.1 binding site mutated, further decreased the −190 mut 1 activity to one-half in the presence of T/EBP/NKX2.1 expression vector. When both binding sites were mutated (−190 mut 3), trans-activating activity by the T/EBP/NKX2.1 was completely abolished. In order to confirm these results, construct −147 mul that has the proximal T/EBP/NKX2.1 binding site mutated, was co-transfected into HeLa cells with and without the TIF-EBP/NKX2. 1 expression plasmid. The mutated binding site almost abolished the trans-activating activity (FIG. 3C). Thus, elements that mediate the transcriptional activation of T/EBP/NKX2.1 are located at position −182 and −120 bp in the 5′ region of the mouse UGRP1 gene.

### EXAMPLE 6

### Characterization of UGRP1 Proteins

The polypeptides of uteroglobin/CCSP family members form a homodimer or heterodimer (Mukherjee et al., Cell Mol Life Sci 55:771-787, 1999). To examine whether three types of UGRP1 polypeptides can form a homodimer, they were tagged with a c-myc epitope (39 amino acids including the linker sequence) at the carboxyl-termini and were individually transfected into COS-1 cells. Immunoblot analyses with anti-c-myc antibody clearly demonstrated that in cell lysate, both polypeptides A and B are mainly present as a dimeric form as seen in non-reducing condition, which is reduced to monomeric form when reducing condition is used. A small amount of monomeric forms of polypeptides A and B were also found in non-reducing conditions. In conditioned medium, only homodimers were detected for polypeptides A and B, indicating that only dimeric forms may be directed to the secretory pathway. In contrast, no band corresponding to polypeptide C was ever detected in either the cell lysate or the medium under any conditions examined, despite the effort to keep the procedures of transient expression and immunoblot analyses consistent for all three polypeptides. This may indicate that the transcript encoding polypeptide C has different translation efficiency and/or the protein is susceptible to degradation.

### EXAMPLE 7

### UGRP1 Expression in Lung Airways and Its Involvement in Inflammation

Polyclonal antibody against mouse UGRP1 was raised using bacterially expressed UGRP1 type A polypeptide. The antibody specificity for UGRP1 was examined by western blotting using c-myc epitope-tagged UGRP1 and uteroglobin/CCSP polypeptides expressed in COS-1 cells, in which anti-UGRP1 antibody reacted with only UGRP1. Wild-type newborn embryo lungs were then subjected to immunohistochemistry using this antibody. Positive immunostaining for UGRP1 was clearly found in the epithelial cells of the trachea, bronchus, and bronchioles whereas the immunostaining for uteroglobin/CCSP was detected in only the bronchus and bronchioles, but not the trachea. These results further indicate that the anti-UGRP1 antibody does not cross-react with uteroglobin/CCSP. Most of UGRP1 immunopositive cells in the bronchioles are Clara cells. The T/ebp/Nkx2.1-null embryo lungs did not have any positive staining as expected.

In order to obtain information regarding UGRP1 function, Northern analyses were performed using RNAs isolated from whole lungs of naive, antigen exposed, antigen exposed and dexamethasone treated, and dexamethasone alone treated animals. Antigen-induced inflammation in BALB/c mice was associated with a significant decrease in UGRP1 expression. The expression of UGRP1 was returned toward normal with dexamethasone treatment. Dexamethasone treatment alone did not change the level of UGRP1 expression. The expression of UGRP2 was also examined, which showed a similar pattern of changes to UGRP1 although the decrease after antigen treatment was not as significant.

Thus, a gene that is mainly expressed in lung, UGRP1, has been cloned. The presence of orthologous and homologous genes in mouse and human suggests that they consist of a new gene family. The UGRP1 amino acid sequence shows sequence similarities to those of the uteroglobin/CCSP gene family of proteins (Mukherjee et al., Cell Mol Life Sci 55:771-787, 1999; Watson et al., Cancer Res 56:860-865, 1996; Mukherjee et al., Ann NY Acad Sci 923, 2000; Margraf et al., Am J Respir Cell Mol Biol 9:231-238, 1993; Parker et al., J Biol Chem 258:12-15, 1983). Uteroglobin/CCSP, the main member of the gene family, is a progesterone-inducible, homodimeric secretory protein expressed in many organs, such as uterus, lung, mammary gland, and prostate (Mukherjee et al., Cell Mol Life Sci 55:771-787, 1999; Mukherjee et al., Ann NY Acad Sci 923, 2000). Western blotting analyses indicate that UGRP1 is a secretory protein which can function as a homodimer as seen in uteroglobin/CCSP. The uteroglobin/CCSP family proteins are characterized by two conserved cysteine residues at the N- and C-terminal regions that are required to form a dimer, and a lysine residue located in between them (Mukherjee et al., Cell Mol Life Sci 55:771-787, 1999; Mukherjee et al., Ann NY Acad Sci 923, 2000). The area containing the conserved lysine residue is called antiflammin in the uteroglobin/CCSP (Mukherjee et al., Cell Mol Life Sci 55:771-787, 1999; Mukherjee et al., Ann NY Acad Sci 923, 2000). The amino acid similarities between UGRP1 and uteroglobin/CCSP family proteins are significant in the areas of signal peptide and antiflammin although overall similarities are low and two conserved cysteine residues are absent in UGRP 1.

UGRP1 mRNA is detected in the lungs of mouse embryos right after the onset of T/EBP/NKX2.1 expression (Lazzaro et al., Development 113:1093-1104, 1991). T/EBP/NKX2.1 is responsible for morphogenesis and cellular differentiation of the distal lung compartments, and appears to be one of the key regulators of early lung development (Minob et al., Dev Biol 209:60-71, 1999; Yuan et al., Dev Dyn 217:180-190, 2000). Northern blot and immunohistochemical analyses indicate that UGRP1 is mainly expressed in lung although expression is also found at lowest detectable levels in the thyroid. Expression in the lung is localized in the epithelial cells of the airways. Analyses of the UGRP1 gene and its transcripts showed that at least three transcripts are produced possibly through an alternative splicing event, in which intron sequence is either spliced, or totally or partially retained in mature mRNAs (Smith et al., Annu Rev Genet 23:527-577, 1989). All the three UGRP1 transcripts are expressed in embryonic lungs with the type A transcript being most abundant.

Despite the sequence similarity, UGRP1 is clearly different from uteroglobin/CCSP as revealed by the following evidence; 1) in the lung, UGRP1 expression is found in the trachea, bronchus and bronchioles whereas uteroglobin/CCSP is expressed only in bronchus and bronchioles but not in the trachea, and 2) the mouse UGRP1 gene is localized on chromosome 18C-D, which is homologous with human chromosome 5q31-q34 whereas all known human members of the uteroglobin/CCSP gene family are localized on chromosome 11q12 (Mukherjee et al., Ann NY Acad Sci 923, 2000). The uteroglobin/CCSP is believed to function as a regulator of inflammation in lung. This is based on several findings such as the inhibition of phospholipase A₂ activity, binding of phospholipase A₂ substrate (phosphatidylcholine/phosphatidylinositol), and the location of the gene in the proximity of other genes involved in regulation of inflammation (Mukherjee et al., Ann NY Acad Sci 923, 2000; Hay et al., Am J Physiol 268: L565-575, 1995; Levin et al., Life Sci 38:1813-1819, 1986; Singh et al., Am J Respir Cell Mol Biol 1517:141-14331, 1997). The antiflammin domain exhibits potent anti-inflammatory and immunomodulatory activities, and appears to be responsible for the phospholipase A₂-inhibitory activity of uteroglobin/CCSP (Mukherjee et al., Cell Mol Life Sci 55:771-787, 1999; Mukherjee et al., Ann NY Acad Sci 923, 2000). A recent report described that uteroglobin/CCSP expression is markedly reduced after acute lung inflammation induced by lipopolysaccharide administration (Arsalane et al., Am J Respir Crit Care Med 161:1624-1630, 2000).

Based on the results of lung-specific expression of UGRP1, the potential anti-inflammatory role described for uteroglobin/CCSP (Mukherjee et al., Cell Mol Life Sci 55:771-787, 1999; Mukherjee et al., Ann NY Acad Sci 923, 2000, Arsalane et al., Am J Respir Crit Care Med 161:1624-1630, 2000), and the likely chromosomal localization of human UGRP1 gene on chromosome 5q31-q34, the region known to contain at least one asthma susceptibility locus (A genome-wide search for asthma susceptibility loci in ethnically diverse populations. The Collaborative Study on the Genetics of Asthma (CSGA). Nat Genet 15:389-392, 1997; Cookson et al., Hum Mol Genet 9:2359-2364, 2000; Postma et al., N Engl J Med 333:894-900, 1995), the association between allergic lung inflammation and the expression of UGRP1 and UGRP2 was examined in vivo. The allergic models used are associated with TH2 cytokine-mediated inflammatory responses in the lung (Mehlhop et al., Proc Natl Acad Sci U S A 94:1344-1349, 1997; Kurup et al., J Immunol 148:3783-3788, 1992). Antigen-induced lung inflammation was associated with decreased expression of UGRP1 and UGRP2. Steroid treatment in vivo increased their expression toward baseline, or to levels found in naive animals. Similar results were observed for uteroglobin/CCSP in vivo after lipopolysaccharide (LPS) induced acute lung inflammation (Arsalane et al., Am J Respir Crit Care Med 161:1624-1630, 2000). LPS caused a marked reduction in uteroglobin/CCSP expression in bronchoalveolar lavage fluid and lung homogenates. In this case, at high LPS concentration, the decrease of uteroglobin/CCSP level was thought to be associated with a reduction of the number of Clara cells that is a consequence of damage to Clara cells secondary to pulmonary inflammation, and possibly with the intravascular leakage of the protein across the disrupted bronchoalveolar blood barrier (Arsalane et al., Am J Respir Crit Care Med 161:1624-1630, 2000). Furthermore, dexamethasone pretreatment failed to prevent the LPS-induced changes in uteroglobin/CCSP levels (Arsalane et al., Am J Respir Crit Care Med 161:1624-1630, 2000). Reduced lung expression of uteroglobin/CCSP was also reported in mice after bacterial infection and human asthmatic patients. The expression pattern of UGRP1 and UGRP2 after antigen treatment appears to be consistent with that observed for uteroglobin/CCSP. The decreased expression of UGRP1 and 2 could be due to a different mechanism(s). Without being bound by theory, it is possible that, UGRP1 and 2 are down-regulated by both TH1 and TH2 inflammatory cytokines that are in turn down-regulated by steroid treatment.

The T/EBP/NKX2.1 was found to trans-activate mouse UGRP1 gene promoter. DNase I footprinting analyses of the promoter region and co-transfection experiments of UGRP1 -luciferase reporter constructs with the T/EBP/NKX2.1 expression plasmid delineated a minimal region of the UGRP1 gene promoter that is sufficient to activate the transcription. This region contains two consensus T/EBP/NKX2.1 binding elements, 5′-CTNNAG-3′ (Bohinski et al., Mol Cell Biol 14:5671-5681, 1994).

Mutation of this motif in the binding sites interfered with T/EBP/NKX2.1 from binding to the site and reduced its ability to activate transcription. Since construct −190 mut 2 showed a larger decrease in activity as compared with the −190 mut 1, it appears that the proximal T/EBP/NKX2.1 binding site is more important for the promoter activity than the distal one, yet both are required for full activity. Electrophoretic mobility shift assays confirmed that T/EBP/NKX2.1 interacts with the two binding sites. The requirement of two T/EBP/NKX2.1 binding sites for full promoter activity has been reported in the Sp-B and C promoters (Bohinski et al., Mol Cell Biol 14:5671-5681, 1994; Kelly et al., J Biol Chem 271:6881-6888, 1996).

Anti-TTF1 monoclonal antibody only slightly supershifted the protein-DNA complex. Without being bound by theory, it is possible that the epitope may be buried once the protein complexes with DNA, thus interfering with a tertiary complex formation. DNase I footprinting protected region IV using bacterially-expressed recombinant T/EBP/NKX2.1 did not produce any specific protein-DNA shifted band when examined by gel shift analyses using NCI-H441 nuclear extracts. This region does not have a typical T/EBP/NKX2.1 binding consensus sequence. It remains a possibility that T/EBP/NKX2.1 binds to the region when no other protein is around as seen in the footprinting analyses. This could explain the incomplete reduction of luciferase activity obtained with the −147 mut construct when co-transfected into HeLa cells with the expression plasmid. The massive amount of expressed T/EBP/NKX2.1 may successfully compete with other proteins to unmask and bind to the region, leading to a slight activation. Other transcription factors such as hepatocyte nuclear factor 3 (HNF-3) family members and the HNF-3/forkhead homologs (HFHs) are known to be involved in the expression of lung-specific genes including SP-B and uteroglobin/CCSP genes (Bohinski et al., Mol Cell Biol 14:5671-5681, 1994; Clevidence et al., Dev Biol 166:195-209, 1994; Kurup et al., J Immunol 148:3783-3788, 1992). HNF-3 and HFH transcription factors may also be involved in the mouse UGRP1 gene promoter activity.

### EXAMPLE 8

### Methods for Human Studies

Cloning and DNA Sequencing

A human UGRP1 genomic DNA was isolated from a human BAC DNA library (Incyte Genomics, St. Louis, Mo.) and Human GenomeWalker™ kit (Clontech, Palo Alto, Calif.). The genomic DNA fragments were sequenced using an ABI prism dye terminator cycle sequencing ready reaction kit and a model 377 DNA sequencer (PE Applied Biosystems, Foster City, Calif.). The transcription start site of the human UGRP1 transcript was determined by SMART™ RACE cDNA amplification kit (Clontech) using 2 μg of human adult lung-total RNA according to the manufacturer's direction. DNA sequence analyses indicated the presence of multiple transcription start sites. Since the majority of clones (twelve out of sixteen) had the exact same sequence, we refer to this site as the major transcription start site.

Chromosomal Mapping

A human UGRP1 probe of an entire BAC genomic clone labeled with biotin or digoxigenin was used for fluorescence in situ hybridization (FISH) of chromosomes derived from methotrexate-synchronized normal peripheral lymphocytes. Conditions of hybridization, detection of hybridization signals, digital-image acquisition, processing and analysis, direct fluorescent signal localization on banded chromosomes were performed as previously described.

Genotyping

Genotyping for the −112G/A polymorphism was performed using a PCR fragment amplified by the following primers; 5′-CCTCCAGATTGCTTTCACAACTGGG-3′ (SEQ ID NO:24) and 5′-CAAAGTGTGATGGCTGCTTTTGCAC-3′ (SEQ ID NO:25). PCR was performed in a 20 μl reaction mixture containing 50 ng of genomic DNA under the following conditions: denaturation at 94° C. for 30 s, annealing and extension at 68° C. for 1 min, for 35 cycles. Amplified DNA fragments were purified and sequenced.

Transfection and Reporter Gene Assays

A 294 bp fragment (from −209 to +85) of human UGRP1 gene promoter was prepared by PCR using two forms (−112G/G and −112A/A) of genomic DNA as a template, each of which was separately subcloned into a NheI-XhoI site of the pGL3-Basic luciferase reporter vector (Promega, Madison, Wis.) to generate the pGL3-112G and pGL3-112A plasmids.

Conditions of culturing human lung adenocarcinoma NCI-H441 cells and the method of transfection are as previously described. Luciferase activity was normalized to β-galactosidase activity and the relative luciferase activity of two human UGRP1 promoter constructs was expressed based on the activity of pGL3-Basic plasmid as 1. Data are the mean value of four independent experiments (duplicate samples)±S.D.

Electrophoretic Mobility Shift Assays (EMSA)

Single-stranded oligonucleotides were annealed to produce double-stranded DNA, either having G or A on both strands at −112 bp (−112G or −112A oligonucleotide). Double-stranded DNA was end-labeled with [α-³²P]dCTP and DNA polymerase Klenow fragment (Life Technologies). Nuclear extracts was prepared from human lung NCI-H441 cells and EMSA carried out as previously described.

Subjects for Case-control Study

Japanese subjects with bronchial asthma were recruited from the pulmonary clinic at the University Hospital. Asthma was diagnosed by the following criteria:

(1) presence of at least two symptoms (recurrent cough, wheezing, or dyspnea);

(2) presence of reversible airflow limitation (15% variability in forced expiratory volume in one second (FEV1) or in peak expiratory flow rate either spontaneously or with an inhaled short-acting beta2-agonist) or increased airway responsiveness to methacholine; and

(3) absence of other pulmonary diseases.

Subjects consisted of a total of 84 asthmatics (40 males, 44 females) and their average age was 46.3+/−16.3 [SD] years old.

Eighty-five control subjects were frequency matched by age and were selected from among normal healthy Japanese volunteers without history of bronchial asthma or other respiratory symptoms. Their average age was 44.8+/−8.9 [SD] years old and consisted of 58 males and 27 females. All participants did not have a family history of asthma and gave informed consent.

Genomic DNA was extracted from peripheral leukocytes isolated from EDTA-anticoagulated blood using a commercially available DNA isolation kit (DnaQuick, Dainippon Pharmaceutical, Co., Osaka, Japan).

### EXAMPLE 9

### Expression of Human UGRP1

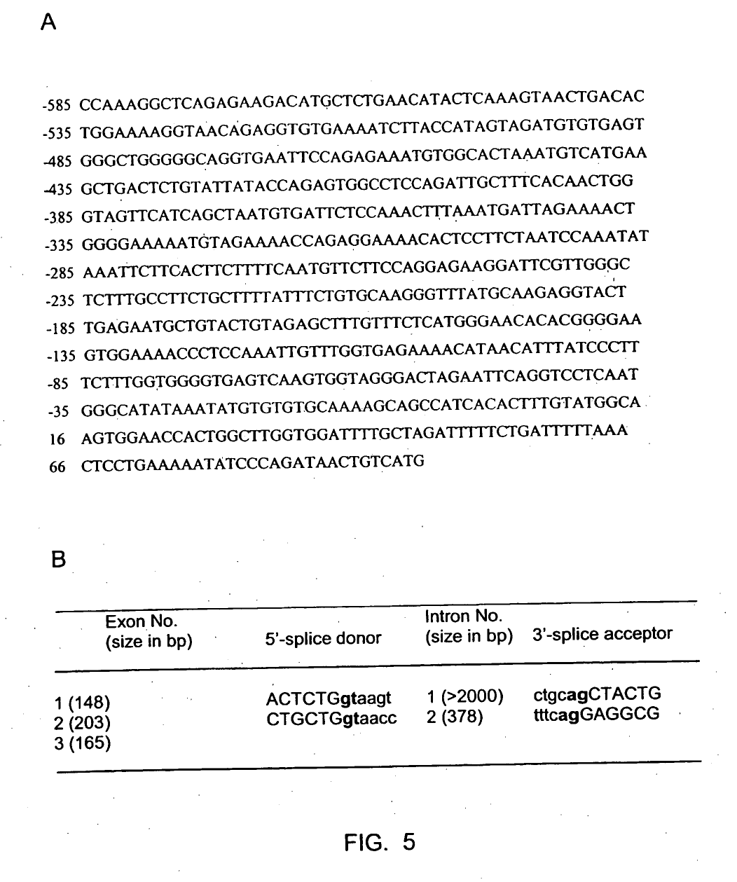

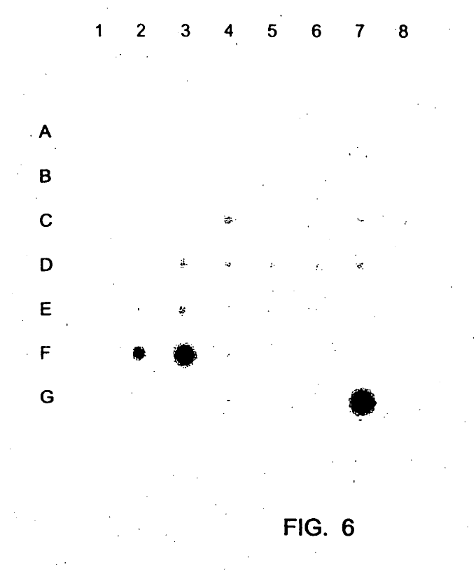

Human UGRP1 gene spans at least 2,900 base pairs in length and consists of three exons with the first intron 5-6-fold longer than the second intron. This structure resembles the structure of orthologous mouse UGRP1 gene. All the exon-intron boundaries demonstrated a consensus sequence for RNA splicing (FIG. 5). The expression of UGRP1 mRNA was detected in only lung and trachea; fetal lung had the highest expression, whereas in adult, higher expression was found in trachea, as compared with a very little expression in lung (FIG. 6).

Human UGRP1 gene was mapped by FISH on chromosomes prepared from normal human peripheral leukocytes. A symmetrical fluorescent signal on sister chromatids was observed in both chromosomes 5 at identical sites in 38 out of 40 metaphases recorded from two separate experiments. The probe had high specificity for this site, as a symmetrical signal was not observed on other chromosomes. In twenty metaphases analyzed by imaging of DAPI G-like banding, the FISH signal was localized on chromosome 5 at region q31-q32. Chromosome 5q31-q34 has been assigned as one of the loci containing asthma susceptible gene(s), which is linked to high total serum IgE level and BHR. The locus includes genes encoding a number of proinflammatory cytokines such as Interleukin (IL)3, IL4, IL5, IL9, IL13 and granulocyte macrophage colony-stimulating factor, and the β₂-adrenergic receptor. The mouse orthologous UGRP1 gene has been mapped to chromosome 18C-D, the region syntenic with the human chromosome 5q31-q32.

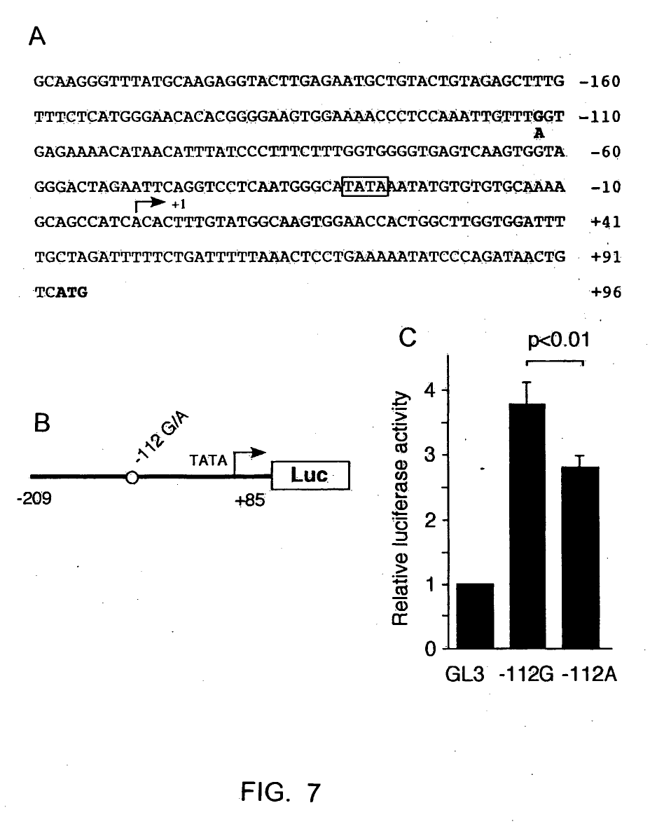

In order to detect sequence variations in human UGRP1 gene that alter the concentration or activity of the coded protein, ultimately contributing to the development of clinical asthma, DNAs from fifty-one randomly selected individuals with or without asthma/allergy (rhinitis) were first screened for possible sequence variation(s) in the coding region and in all the exon-intron boundaries. No variations were found. A 585-bp upstream region of the gene was then screened using the same DNA samples. Two out of fifty-one demonstrated a homozygous guanine to adenine substitution at position −112 relative to the transcription start site of the gene (FIG. 7A).

In order to examine whether the −112G to A polymorphism influences promoter activity of the UGRP1 gene, transfection studies were conducted using lung adenocarcinoma NCI-H441 cells. Luciferase activities were compared between two constructs containing either G or A at −112 bp in the UGRP1 gene promoter region (FIG. 7B). Significantly lower luciferase activity was observed for the −112A construct, as compared with the −112G construct (24% decrease; P<0.01) (FIG. 7C). These results indicate that the −112A allele is associated with the decreased transcriptional activity of UGRP1 gene in lung cells.

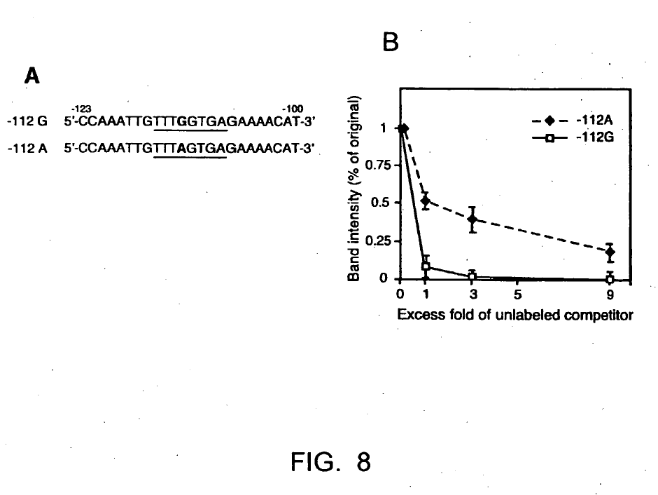

Electrophoretic mobility shift analysis using nuclear extracts prepared from NCI-H441 cells was used to examine whether the nucleotide substitution at −112 bp affects interaction of a nuclear protein(s) with this region. For these studies, 24-bp double-stranded oligonucleotide probes were used. These probes have a sequence from −123 to −100 bp of the UGRP1 gene promoter, with either G or A at −112 bp (−112G or −112A oligonucleotide). A single band due to a specific DNA-protein interaction was obtained at the same mobility with both oligonucleotide probes. To determine whether this particular nuclear protein binds preferentially to one of the two oligonucleotides, a series of competition assays were performed, in which radiolabeled −112G probe was competed against unlabeled −112G or −112A oligonucleotide (FIG. 8). The unlabeled −112G oligonucleotide was a more efficient competitor, having an approximately two-fold higher affinity for a specific DNA-protein complex formation, as compared with that of the −112A oligonucleotide. These results indicate that the A residue at −112 bp interferes with a specific binding property of this particular nuclear protein to the site around −112 bp.

Computer analysis was performed using TF(transcription factor) Search in order to identify a nuclear protein that appears to bind to the sequence around −112 bp and contributes to the transcriptional activation of human UGRP1 gene. The CCAAT/enhancer binding protein (C/EBP) consensus sequence (T[G/T] TGG[A/T]NA) was identified as a candidate transcription factor. There are several C/EBP transcription factors, known as C/EBPα, β, χ, and δ that are potential candidate factors for this interaction. When the C/EBP consensus binding sequence (TTGCGCAAT) was used as a competitor in an electrophoretic gel mobility shift analysis using NCI-H441 cell nuclear extract and the −112G oligonucleotide, no competition was observed for a specific DNA-protein complex formation, suggesting that a nuclear protein binding to the sequence around −112 bp is not a member of the C/EBP family. Thus, it appears that an unknown nuclear protein binds to a sequence similar to the C/EBP binding site located around −112 bp in the human UGRP1 gene promoter and activates transcription of the gene. The G to A point mutation at −112 bp in the human UGRP1 gene promoter decreases the affinity of this nuclear protein to bind to the binding site around −112 bp, which results in decreased transcriptional activity, ultimately leading to decreased expression of UGRP1 protein.

### EXAMPLE 10

### Case Controlled Studies

A case controlled study was performed to examine whether the −112A allele is associated with an increased risk of asthma. This analysis included a total of 169 Japanese subjects, 84 with asthma and 85 without asthma; 98 (58.0%) were male. The mean age of the 169 subjects was 45.6 years old (range 18-81). The mean age was similar between cases and controls (46.3 vs. 44.8 years; P=0.70 by Kruskal-Wallis test). The mean IgE level was significantly higher among asthmatic subjects than among non-asthmatic control subjects (679.4 IU/ml vs. 140.4 IU/ml; P=0.0001 by Kruskal-Wallis test). The IgE level did not differ by gender (443.4 IU/ml in men and 356.0 IU/ml in women; P=0.69 by Kruskal-Wallis test).

The allele frequency of the −112A variant in the UGRP1 gene among asthmatic subjects was 22.0%, compared with 10% among non-asthmatic subjects (P=0.003 by χ² test) (Table 1).

TABLE 1
Frequency of −112A Allele in the UGRP1 Gene among
169 Japanese Subjects with and without Asthma.

| | Genotype | | | A allele |
|---|---|---|---|---|
| Subjects | −112G/G | −112G/A | −112A/A | frequency |
| Control (n = 85) | 70 | 13 | 2 | 10.0% |
| Asthma (n = 84) | 50 | 31 | 3 | 22.0% |

The proportion of subjects with the A variant (G/A and A/A genotype combined) was 40.5% (34 of 84) among asthmatic subjects, compared with 17.6% (15 of 85) among non-asthmatic subjects (P=0.001 by χ² test). Taken together, a person with G/A or A/A genotype was 4.1 times more likely to be asthmatic, compared with those with G/G genotype. Every 100 IU/ml increase in IgE level increases the risk of asthma by 1.9-fold, on average (Table 2).

TABLE 2
The Association of Asthma with UGRP1 Variant and IgE Level.

| Variable | OR | 95% CI |
|---|---|---|
| G/A or A/A genotype | 4.11 | 1.51-11.17 |
| IgE | 1.90 | 1.41-2.58 |

OR, odds ratio; CI, confidence interval.
Logistic-regression models include both variables shown, adjusted for age, sex, and smoking status. Age and IgE level were continuous variables. OR for IgE indicates that every increase of 100 IU/ml in IgE level elevates the risk of asthma 1.9-fold. Reference group is the wild-type (G/G).

UGRP1 is similar to Clara cell secretory protein (CCSP/CC16). Both UGRP1 and CCSP/CC16 genes have three short exons with 1st intron longer than the 2nd. Their protein products are homodimeric and secretory, having amino acid sequences similar to each other (25% identity in mouse). In mouse lung, UGRP1 is expressed in trachea, in addition to bronchus and bronchioles, whereas CCSP/CC16 is expressed only in the bronchus and bronchioles. Further, decreased UGRP1 expression was observed in antigen-treated inflamed mouse lungs, similar to what was reported for CCSP/CC16, where CCSP/CC16 protein expression was decreased after induction of airway inflammation in animals, and in human asthmatic individuals. It is believed that CCSP/CC16 functions as an anti-inflammatory agent, based on numerous in vivo and in vitro studies. Thus, CCSP/CC16 modulates the production and/or the activity of various mediators of the inflammatory response, including phospholipase A₂ (PLA₂), interferon-γ and tumor necrosis factor-α. CCSP/CC16 also inhibits chemotaxis and phagocytosis of monocyte and polymorphonuclar neutrophils. Further support comes from the CCSP/CC16 (−/−) knockout mouse, which exhibits increased inflammation, neutrophil infiltration, and proinflammatory cytokine production in the lungs after administration of adenovirus.

UGRP1 may play a role as an anti-inflammatory agent like CCSP/CC16 in the modulation of pulmonary inflammation. In this respect, it is interesting to note that UGRP1 gene is localized in the area where many proinflammatory cytokine genes are located, and the association of the −112A polymorphism with asthma is independent of the increase in IgE level. Provided that UGRP1 has an anti-inflammatory function, the reduced level of UGRP1 due to −112A polymorphism may contribute to airway inflammation and ultimately the development of asthma.

The art of the present disclosure can be modified in arrangement and detail without departing from such principles. In view of the many possible embodiments to which the principles can be applied, it should be recognized that the illustrated embodiments are only examples and should not be taken as a limitation on the scope of the invention. Rather, the scope is in accord with the following claims. We therefore claim all that comes within the scope and spirit of these claims.

We claim:

1. A nucleic acid sequence comprising SEQ ID NO:1 or SEQ ID NO:19, or a conservative variant thereof, operably linked to a heterologous nucleic acid.

2. A vector comprising the nucleic acid sequence of claim 1.

3. The vector of claim 2, wherein the vector is a viral vector.

4. A host cell transfected with the vector of claim 2.

5. The host cell of claim 4, wherein the host cell is a mammalian cell.

6. The host cell of claim 4, wherein the host cell is a human cell.

7. The nucleic acid sequence of claim 1, wherein the heterologous nucleic acid encodes a polypeptide.

8. The nucleic acid of claim 1, wherein the heterologous nucleic acid is a antisense molecule or a ribozyme.

9. A nucleic acid sequence consisting essentially of SEQ ID NO:1.

10. A nucleic acid sequence consisting essentially of SEQ ID NO:19.

11. A method for diagnosing or predicting a predisposition to develop a respiratory disorder in a subject, comprising:
detecting a polymorphism in a UGRP1 promoter in said subject, wherein detection of the polymorphism is indicative that the subject has a respiratory disorder, or a predisposition to develop the respiratory disorder.

12. The method of claim 11, wherein the polymorphism is a G to A transition at position −112 in SEQ ID NO:1 or a conservative variant thereof.

13. The method of claim 12, wherein the polymorphism is in region −209 to +96 bp of SEQ ID NO:1.

14. The method of claim 12, wherein the respiratory disorder is asthma.

15. The method of claim 11, wherein the detection utilizes RT-PCR.

16. The method of claim 11, wherein the detection utilizes a restriction enzyme polymorphism.

17. The method of claim 11, further comprising determining whether the subject is homozygous or heterozygous for the polymorphism.

18. A method of determining the prognosis of a subject having or suspected of having a respiratory disorder, comprising:
detecting a polymorphism in the UGRP1 promoter in a sample obtained from the subject, wherein the detection of the polymorphism in the sample is indicative of the prognosis of the respiratory disorder in the subject.

19. The method of claim 18, wherein the polymorphism is a G to A transition at position −112 in SEQ ID NO:1 or a conservative variant thereof.

20. The method of claim 19, wherein the polymorphism is in region −209 to +96 bp of SEQ ID NO:1.

21. The method of claim 18, wherein the respiratory disorder is asthma.

22. The method of claim 18, wherein the detection utilizes RT-PCR.

23. The method of claim 18, wherein the detection utilizes a restriction enzyme polymorphism.

24. A method of predicting a predisposition to having a respiratory disorder in a subject, comprising
obtaining a test sample of DNA containing an UGRP1 promoter sequence of the subject; and
determining whether the subject has a polymorphism in the UGRP1 promoter sequence, wherein the presence of the polymorphism indicates the subject has the predisposition to the respiratory disorder.

25. The method of claim 24, wherein determining whether the subject has the polymorphism comprises using restriction digestion, probe hybridization, nucleic acid amplification, or nucleotide sequencing.

26. The method of claim 25, wherein determining whether the subject has the polymorphism comprises using nucleic acid amplification.

27. The method of claim 26, wherein determining whether the subject has the polymorphism comprises using polymerase chain reaction nucleic acid amplification.

28. The method of claim 24, wherein the polymorphism is a G to A transition at position −112 in the UGRP1 promoter sequence.

29. The method of claim 28, wherein the UGRP promoter sequence has a sequence as set forth as SEQ ID NO:1.

30. The method of claim 28, wherein the respiratory disorder is asthma.

31. A method of predicting predisposition to a respiratory disorder in a subject, comprising:
obtaining from the subject a test sample of DNA comprising an UGRP1 promoter sequence;
contacting the test sample with at least one nucleic acid probe for an UGRP1 promoter sequence polymorphism that is associated with increased predisposition to the respiratory disorder in a subject to form a hybridization sample;
maintaining the hybridization sample under conditions sufficient for specific hybridization of the UGRP1 promoter sequence with the nucleic acid probe; and
detecting whether there is specific hybridization of the UGRP1 promoter sequence with the nucleic acid probe, wherein specific hybridization of the UGRP1 promoter sequence with the nucleic acid probe indicates increased predisposition to the respiratory disorder in the subject.

32. The method of claim 31, wherein the polymorphism is a G to A transition at position −112 in the UGRP1 promoter sequence.

33. The method of claim 31, wherein the probe is present on a substrate.

34. The method of claim 33, wherein the substrate is a nucleotide array.

35. The method of claim 32, wherein the respiratory disorder is asthma.

36. A kit for use in diagnosing an increased predisposition to a respiratory disorder in a subject, comprising a probe that specifically hybridizes to an UGRP1 promoter sequence polymorphism that is associated with the increased predisposition to the respiratory disorder.

37. The kit of claim 36, wherein the hybridization of the probe is used to detect a polymorphism that is a G to A transition at position −112 in the UGRP1 promoter sequence.

38. A nucleic acid probe that specifically hybridizes to a human UGRP1 polymorphism.

39. The nucleic acid probe according to claim 38 wherein the probe hybridizes to a G to A transition at position −112 in the UGRP1 promoter sequence.
